(19) United States
(12) Patent Application Publication
(10) Pub. No.: US 2025/0057939 A1
(43) Pub. Date: Feb. 20, 2025

(54) FREEZE-DRIED VIRAL COMBINATION VACCINE COMPOSITIONS AND PROCESS FOR PREPARATION THEREOF

(71) Applicants: Serum Institute Of India Private Limited, Pune Maharashtra (IN); Codagenix Inc., Farmingdale, NY (US)

(72) Inventors: Rajeev Mhalasakant DHERE, Pune Maharashtra (IN); Leena Ravindra YEOLEKAR, Pune Maharashtra (IN); Rajeev MERLA, Pune Maharashtra (IN); John Robert COLEMAN, Blauvelt, NY (US)

(21) Appl. No.: 18/690,297

(22) PCT Filed: Sep. 8, 2022
(86) PCT No.: PCT/IN2022/050805

§ 371 Date: Mar. 8, 2024

(30) Foreign Application Priority Data
Sep. 8, 2021 (IN) 202121040801

(51) Int. Cl. A61K 39/215; A61K 9/19; A61K 39/00; A61K 39/165; A61K 39/20; A61K 39/39; A61K 47/18; A61K 47/26; A61K 47/42; A61P 37/04

(52) U.S. Cl. CPC A61K 39/215; A61K 9/19; A61K 39/165; A61K 39/20; A61K 39/39; A61K 47/183; A61K 47/26; A61K 47/42; A61P 37/04; A61K 2039/5252; A61K 2039/5254; A61K 2039/5256; A61K 2039/55505; A61K 2039/55561; A61K 2039/55566; A61K 2039/55572; A61K 2039/55577

(57) ABSTRACT

The present disclosure relates to field of lyophilized/freeze-dried viral combination composition/formulation and methods for manufacturing and obtaining the composition comprising at least three live attenuated virus selected from a group of Coronavirus, Measles virus and Rubella virus; and stabilizers comprising of at least one carbohydrate, at least one amino acid and at least one hydrolyzed protein. The said lyophilized/freeze-dried viral combination composition/formulation is a vaccine composition that preserves the desired characteristics of each virus, including stability and immunogenicity. The composition can be safely administered subcutaneously as a combination vaccine composition such that the immunogenicity of each of the measles, rubella and SARS-CoV-2 is not inferior to that observed for each of the three viruses when administered as individual vaccines and is found to be equivalent or improved as compared to immunogenicity of SARS-CoV-2 vaccine given intranasally. The purification process is devoid of chromatography steps.

### FIELD

The present disclosure is related to biotechnology, virology, medicine. It concerns viral vaccines manufacturing, more particularly, it relates to a lyophilized/freeze-dried viral combination composition/formulation comprising of Coronavirus (SARS-CoV-2), Measles & Rubella antigens/immunogens and the methods of preparing the same. The present disclosure further relates to an improved methodology in the field of combination vaccine production.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Epidemics and pandemics caused by infectious agents have been occurring for centuries, causing major disruptions, with varying morbidity and mortality. Recent times have seen multiple challenges from acute virus infections, including severe acute respiratory syndrome (SARS), Middle East respiratory syndrome (MERS), Hendra, Nipah, and Ebola. Coronavirus (CoV) an infectious agent has been one of the major players in the history of epidemics and pandemics. The ripple effect of the COVID-19 outbreak in 2019 has brought major challenges to worldwide health systems and has far-reaching consequences on the global economy.

As it has been found for novel betacoronavirus severe acute respiratory syndrome coronavirus 2 (SARS-CoV-2) the containment of the infection is not possible, hence there is a need to understand and plan for the transition from pandemic to endemic state and continued circulation, with the question of how the severity of infection with severe acute respiratory syndrome coronavirus 2 (SARS-CoV-2) may change in the years ahead. Once the endemic phase is reached and primary exposure of SARS-CoV-2 is restricted to childhood a different outcome for an emergent coronavirus that causes severe disease in children is a possibility. (Refer: Lavine et al., (2021) Science 371 (6530), 741-745). Hence, these results reinforce the importance of continuing vaccination in the endemic phase. The use of vaccines has played an important role in the elimination of a wide variety of infectious diseases.

To reduce the spread of COVID-19, many states and localities issued stay-at-home orders, limiting movement outside the home to essential activities. The COVID-19 pandemic had huge impact on pediatric vaccination compliance around the world. Parental concerns about potentially exposing their children to COVID-19 during routine pediatric vaccine administration among children's has contributed to the decline in routine pediatric vaccine administration among children's and their communities face increased risks for outbreaks of vaccine-preventable diseases. As social distancing requirements are relaxed, children who are not protected by vaccines will be more vulnerable to diseases such as measles, mumps, rubella and pertussis.

SARS-CoV-2 viruses are particularly dangerous for the elderly and those with underlying medical conditions such as chronic kidney disease, chronic obstructive pulmonary disease, being immunocompromised from a solid organ transplant, obesity, serious heart conditions, sickle cell disease and type 2 diabetes mellitus. The world continents have confirmed a total of 1,202,320 confirmed COVID-19 cases: (51.2%) in Europe, (27.7%) in North America, (17.9%) in Asia, (1.96%) in South America and at less number of confirmed COVID-19 cases in Africa and Australia which was accounted 0.8% and 0.5%, respectively. We estimated that 349 million (186-787) people (4% of the global population) are at high risk of severe COVID-19 and would require hospital admission if infected. The share of the population at increased risk was highest in countries with older populations, African countries with high HIV/AIDS prevalence, and small island nations with high diabetes prevalence. Estimates of the number of individuals at increased risk were most sensitive to the prevalence of chronic kidney disease, diabetes, cardiovascular disease, and chronic respiratory disease.

Measles is a disease caused by a Paramyxovirus of the genus Morbillivirus. Measles infection runs a devastating course in children in developing countries, where the mortality rates can be as high as 2% to 15%. Pneumonia is the most common severe complication from measles and is associated with the greatest number of measles-associated deaths. The rash is intense and often hemorrhagic; it resolves after marked desquamation. Inflammation of the mucosa leads to stomatitis and diarrhea. There are other severe complications when the disease affects the brain. Measles outbreaks continue across Africa and Europe, with children under the age of five most affected. More than 95% of measles deaths occur in low-income countries with weak health systems. The region of the Americas, which was verified to have eliminated measles in 2016, lost its elimination status in 2018. The global number of reported measles cases more than doubled in only one year from 170,000 in 2017 to 350,000 in 2018. This upward trend in cases continued into 2019 with several countries experiencing large outbreaks of Measles. In 2019, the Democratic Republic of Congo, Ukraine and Brazil reported 333,017, 57,282, and 18,203 confirmed cases of measles, respectively while Chad reported over 26,600 suspected cases. Vaccination coverage remains low or very low in several countries. In 2019, 7 countries had MCV1 (First dose of measles-containing vaccine) coverage below 50% and 23 had coverage below 70%, indicating that 30-50% of children in these countries had not received any doses of measles vaccine through the routine service delivery mechanisms. Measles kills over 568 people a day, mostly children. 30% of children affected with congenital rubella syndrome die. Over 140,000 people lost their lives to measles in 2018. Measles infections can cause subacute sclerosing panencephalitis (SSPE).

Rubella is a disease caused by a Togavirus of the genus Rubivirus. Usually, a rash on the face and neck develops within 2 weeks after exposure to the virus. The volume of glands increases and subjects experience fever, malaise, and conjunctivitis. Rubella is thought of a benign disease, but complications including brain damages might occur in some subjects. Rubella for millions of mothers and their children in low-income countries, it poses an ongoing danger. When a woman is infected with the rubella virus early in pregnancy, she has a 90% risk of passing the virus on to her foetus. This can cause miscarriage, stillbirth or severe birth defects known as congenital rubella syndrome (CRS). Every year, more than 100,000 babies are born with CRS—the majority in Africa and South-East Asia. Although the rubella vaccine has been available since the 1970s, it is still underused in these regions.

Multiple clinic visits of adults & pediatric population for immunization against SARS CoV2 & MR (Measles-Rubella) increases the complexity of the healthcare and raises specific issues such as compliance, particularly in those areas of the world where healthcare facilities are not regularly available. Consequently, there is a desire to have a combination vaccine for the SARS-CoV-2 and MR to enhance compliance. So far, no approach has been developed to produce a vaccine able to induce immunity against Measles, Rubella combined with immunity against SARS CoV 2.

Hence it is important to develop a pediatric & adult vaccine in combination with existing SARS-CoV-2 vaccine to avoid additional injection and clinic visit.

A combination vaccine combining multiple vaccines into a single shot can provide immunogenicity against multiple diseases and is always advantageous over the monovalent vaccine since it reduces the number of shots given, reduced complications associated with multiple injections, reduces the administration and production costs, decreased costs of stocking, reduced risk of delayed or missed vaccinations and improves the patient compliance by reducing the number of separate vaccinations.

However, there are multiple technical challenges in maintaining immunogenicity and safety when combining vaccines. Main challenge in combination vaccine development is the risk that the efficacy or safety of the combination would be less than that seen with the administration of the vaccines separately. New combinations cannot be less immunogenic, less efficacious, or more reactogenic than the previously licensed uncombined vaccines. Immunological, physical, and/or chemical interactions between the combined components have the potential to alter the immune response to specific components. Furthermore, if the vaccines to be combined have differing immunization schedules, consolidation of these should also not negatively affect immunogenicity, efficacy, or safety. Finally, and ideally, the many advantages of combination vaccines should not be achieved at the cost of reduced product stability. From a practical standpoint, uncommon transport and storage conditions and complicated bedside mixing could hamper the development of a combination vaccine.

The vaccination strategy can greatly influence the immunogenicity, efficacy, and safety of a vaccine. The many factors impacting the efficacy of a vaccine can be broadly divided into three categories: (1) features of the vaccine itself, including immunogen design, vaccine type, formulation, adjuvant use, and dosing; (2) individual variations among vaccine recipients such as gender, age, developmental stage, nutrition status, and pre-existing immune conditions; and (3) vaccine administration-related parameters including vaccination approach, delivery route, and method of administration, number of immunizations, immunization site, and intervals between administrations and use of prime/boost regimens and vaccine modulators. Conventional route of administration of vaccine approaches include mucosal routes (oral, intranasal, pulmonary, rectal or vaginal) and parenteral routes (subcutaneous (sc), intradermal (id) or intramuscular (im) inoculation), and the choice of one strategy over the other depends on the type of vaccine and protective immunity needed to conquer the disease based on the route of infection and transmission.

MR vaccine has been recommended in infants, children above 10 years, and adolescents, and has an excellent track record of safety and efficacy. Both viruses are live attenuated virus strains given via subcutaneous route. Subcutaneous injection has certain advantage over intramuscular injections most preferably it is much easier to self-administer and requires little to no training hence improved convenience, safety, and cost. As subcutaneous tissue does not have a rich blood supply, and absorption of drugs delivered via that route is therefore slower than via the intramuscular route (Dougherty and Lister, 2015). Therefore, it is highly effective in administering vaccines, which require continuous delivery at a low dose rate. The slow injection rate resembles the situation at which active component is gradually released. Further, combination respiratory vaccine containing SARS-CoV-2 and influenza vaccine antigen combined in a single shot have been reported to be under clinical trials.

Previously it was contemplated that CDX-005 (Codagenix's Live Attenuated SARS-CoV-2 virus strain developed through codon de-optimization of the spike protein), if administered intranasally, will be easier to administer and likely more palatable to vaccine recipients. Also the formulation comprising of sucrose (5%), glycine (5%) & surfactants (Polysorbate) has been disclosed for CDX-005 previously. Modema mRNA-1273 (RNA), Pfizer/BioNTech BNT162b2 (RNA), Oxford/AstraZeneca AZD1222, Serum Institute of India—Covishield (Oxford/AstraZeneca formulation) are few of the licensed/approved SARS-CoV-2 vaccines that comprise of sucrose as one of the formulation components. Janssen (Johnson & Johnson) Ad26.COV2.S, Oxford/AstraZeneca AZD1222 & Serum Institute of India—Covishield (Oxford/AstraZeneca formulation) are few of the licensed/approved SARS-CoV-2 vaccines that comprise of Polysorbate 80 as one of the formulation components.

The typical non-ionic surfactants used in pharmaceutical formulations include Triton™ X-100, Pluronic® F-68, F-88, and F-127 (poloxamers), Brij 35 (polyoxy-ethylene alkyl ether), polyoxyl stearate 40, Cremophor® EL, and alpha-tocopherol TPGS. Each of these surfactants have a common fact, in that they all contain polyoxyethylene moieties and thus to a greater or lesser extent, exhibit a similar problem, in that the polyoxyethylene moiety auto oxidizes to produce reactive peroxides, which causes an increase in unwanted protein immunogenicity. (Refer Edward T. Maggio et al; Polysorbates, peroxides, protein aggregation, immunogenicity—a growing concern; Journal of Excipients and Food Chemicals 3(2):46-53; 2012). Polysorbates are prone to degradation by oxidation and hydrolysis with hydrolysis being induced either chemically or enzymatically. Polysorbate may also be auto-oxidized by temperature, light or transition trace metals, and the resulting peroxide formation may induce protein oxidation, whereas the acid produced may lead to a decrease in solution pH. PS80 degraded via hydrolysis led to slower surface adsorption rate, and the free fatty acid release from hydrolysis also forms insoluble particles, negatively impacting protein quality and stability. Polysorbate 80 has also been causally linked with an increased risk of blood clots, stroke, heart attack, heart failure, and of tumor growth or recurrence in patients with certain types of cancer.

Lyophilization is a common mode of stabilization of vaccines. However, lyophilization causes loss in virus potency. Vaccines lose potency over time and the rate of potency loss is temperature-dependent. Live viruses are susceptible to osmotic, thermal and vacuum shocks. Enveloped viruses possess a lipid bilayer, which is considered as the less stable virus component because of its high fragility. Live viruses are susceptible to various stresses during lyophilization steps like freezing, primary drying, secondary drying that could affect the physico-chemical stability of viruses. Owing to their structure, loss of potency during freeze-drying can be due to protein destabilization (e.g. unfolding, degradation, and aggregation), nucleic acid degradation, lipid layer alteration (e.g. phase transition, mechanical damage) and stresses related to changes in the internal (ice formation) and external (pH and osmolarity change) virus environment. The dehydration step of lyophilization results in collapse of the hydrogen bond structure of proteins which is accompanied with increased mobility of amino acid components of virus epitopes. It has been reported that in some cases lyophilization causes upto 40% loss in virus potency. Though a lot of information is available on stress mechanisms and stabilization strategies of pharmaceutical peptides, proteins and DNA during lyophilization, due to the molecular complexity of viruses, different destabilization pathways and lack of analytical techniques permitting measurement of physico-chemical changes in the antigen's structure during and after lyophilization mean that viruses constitute a particular lyophilization challenge. The destabilization mechanisms as well as protection mechanisms for live, attenuated viral vaccines during lyophilization are not well known.

Hence it is important to develop a lyophilized/freeze dried live attenuated viral combination vaccines for pediatric & adults comprising Measles-Rubella antigens in combination with existing SARS-CoV-2 vaccine to avoid additional injection and clinic visit and which may prove useful in giving high level, long term immunity in children.

### Objects

Some of the objects of the present disclosure, which at least one embodiment herein satisfies, are as follows:

An object of the present disclosure is to ameliorate one or more problems of the prior art or to at least provide a useful alternative.

An object of the present disclosure is to provide a lyophilized/freeze-dried viral combination composition wherein, said virus is selected from a group of but not limited to a live attenuated virus (LAV), an inactivated virus, a chimeric virus, or a recombinant virus.

Yet another embodiment is that said lyophilized/freeze-dried viral combination composition is a vaccine composition that preserves the desired characteristics of each virus, including stability and immunogenicity.

Another object of the present disclosure is to provide a lyophilized/freeze-dried live attenuated viral combination vaccine composition/formulation comprising at least three viruses selected from a group consisting of a live attenuated Measles, Rubella and Coronavirus (SARS-CoV-2 virus).

Another object of the present disclosure is to provide a lyophilized viral combination vaccine composition comprising live attenuated Measles, live attenuated Rubella and live attenuated Coronavirus (SARS-CoV-2 virus) combined together to be given in a single shot and which meets the criterion for the seroprotection for each of the said immunogenic components.

Another object of the present disclosure is to provide methods for manufacturing such lyophilized viral vaccine composition.

Still another object of the present disclosure is to provide a method of vaccinating a host comprising parenteral immunization.

Still another object of the present disclosure is to provide a kit comprising a first container containing a lyophilized (freeze-dried) viral combination vaccine composition and a second container containing an aqueous solution optionally saline or WFI (water for injection) for the reconstitution of the lyophilized (freeze-dried) combination vaccine composition/formulation comprising a live attenuated Measles, live attenuated Rubella and live attenuated SARS-CoV-2 virus.

In various embodiments, the SARS-CoV-2 coronavirus can be a SARS-CoV-2 variant. In various embodiments, said SARS-CoV-2 variant can be selected from the group consisting of U.K. variant, South Africa variant, and Brazil variant, all as of 5 Sep. 2022. The live attenuated Coronavirus vaccine strain of the invention could be the WA/1 strain, an alpha variant, delta variant, delta plus variant, beta variant, gamma variant, delta AY.3 variant, lambda variant, Omicron variant, Epsilon (B.1.427 and B.1.429); Eta (B.1.525); Iota (B.1.526); Kappa (B.1.617.1) 1.617.3; Mu (B.1.621, B.1.621.1); Zeta (P.2) or any other SARS-COV-2 strain that is at least 90% genetically similar to the WA/1 strain.

Other objects and advantages of the present disclosure will be more apparent from the following description, which is not intended to limit the scope of the present disclosure.

### SUMMARY

Applicant has found that a live attenuated measles-rubella (MR) vaccine and a SARS CoV 2 vaccine comprising Live Attenuated SARS-CoV-2 virus strain developed through codon de-optimization of the spike protein can be safely administered subcutaneously as a combination vaccine composition such that the immunogenicity of each of the measles, rubella and SARS-CoV2 is not inferior to that observed for each of the three viruses when administered as individual vaccines and is found to be equivalent or improved as compared to immunogenicity of SARS-CoV-2 vaccine given intranasally. Applicant has found that the formulation preferably is a lyophilized formulation comprising of SARS-CoV-2 vaccine with Measles-Rubella antigens along with sorbitol, gelatin, histidine, alanine, tricine, arginine & lactalbumin hydrolysate is stable, immunogenic. Sorbitol preserves the structural integrity (native like structure) of virus and prevents aggregation of viruses. Lactalbumin hydrolysate prevents adsorption of the viruses onto the walls of the vessel and promotes desorption done previously. Lactalbumin hydrolysate stabilizes proteins through a variety of mechanisms such as preferential hydration, direct binding, buffering, and antioxidation. Said composition is devoid of surfactants like polysorbate. Further said composition does not require any adjuvant.

### DETAILED DESCRIPTION

Although the present disclosure may be susceptible to different embodiments, and following detailed discussion, with the understanding that the present disclosure can be considered an exemplification of the principles of the disclosure and is not intended to limit the scope of disclosure to that which is illustrated and disclosed in this description.

Embodiments are provided so as to thoroughly and fully convey the scope of the present disclosure to the person skilled in the art. Numerous details are set forth, relating to specific components, and processes, to provide a complete understanding of embodiments of the present disclosure. It will be apparent to the person skilled in the art that the details provided in the embodiments should not be construed to limit the scope of the present disclosure. In some embodiments, well-known composition, well-known processes, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the process of the present disclosure is not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure. The present disclosure provides a vaccine composition and a process for preparing the same.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non limiting example of a feature that is intended to be combined with any other feature, or combination of features, described herein without having to list every possible combination. Such features or combinations of features apply to any of the aspects of the invention.

As used herein the terms “Freeze-drying” or “lyophilize” or “lyophilization” involves lyophilization and refers to the process by which a suspension is frozen, after which the water is removed by sublimation at low pressure.

The present disclosure envisages a lyophilized/freeze-dried viral combination vaccine composition/formulation wherein, post-reconstitution the composition preserves the desired characteristics of the virus, including stability and immunogenicity.

According to an embodiment of the present disclosure, the lyophilized/freeze-dried viral combination vaccine composition/formulation may comprise of at least three viruses; at least one carbohydrate; at least one amino acid; and at least one hydrolyzed protein.

According to an embodiment of the present disclosure, lyophilized/freeze-dried vaccine composition/formulation may comprise of at least three viruses selected from a group consisting of poxvirus (e.g. orthopoxviruses; avipoxviruses), morbillivirus (e.g. measles), mumps virus, rubella virus, alphavirus (e.g. sendai virus, sindbis virus and semliki forest virus (SFV), ross river virus, encephalitis virus, flavivirus (e.g. yellow fever virus, dengue virus, Japanese encephalitis (JE) virus, Kunjin virus, West Nile (WN) virus, tick-borne encephalitis (TBE) virus, St. Louis encephalitis virus, Murray Valley encephalitis virus, Zika virus), rhabdovirus (e.g. vesicular stomatitis virus (VSV)), retrovirus (e.g. RNA tumor viruses), adenovirus (e.g. human adenovirus, bovine adenovirus, a canine adenovirus, a non-human primate adenovirus, a chicken adenovirus, or a porcine or swine adenovirus), adeno-associated viruses, lentiviral (e.g., human immunodeficiency viruses (HIV), simian immunodeficiency virus (SIV), and feline immunodeficiency virus (FIV)), herpes simplex virus, cytomegalovirus, picornavirus (e.g. Rhinovirus, Poliovirus etc), baculovirus vectors (autographacalifornica multiple nucleopolyhedrovirus (AcMNPV), hepatitis B virus (HBV), rubulavirus (new castle disease virus), parainfluenza virus, influenza virus, respiratory syncytial virus (RSV), human metapneumovirus (hMPV), respiratory Coronavirus (CoV), Ebola, Marburg, Nipah, Chikungunya, Rotavirus, Human papilloma virus, Herpes simplex, Hepatitis A, Hepatitis C, Hepatitis B, Hepatitis E, Poliovirus, Variola Virus (e.g. smallpox, Monkeypox) and Varicella virus antigens.

According to an embodiment of the present disclosure, lyophilized/freeze-dried viral combination vaccine composition/formulation may comprise of at least three viruses selected from a group consisting of Coronavirus vaccine antigen, Measles virus vaccine antigen and Rubella virus vaccine antigen.

According to an embodiment of the present disclosure, a lyophilized/freeze-dried viral combination vaccine composition comprising Measles, Rubella and Coronavirus (SARS-CoV-2 virus) combined together to be given in a single shot, suitable for the prevention and treatment of more than one disease state and which meets the criterion for the seroprotection for each of the said immunogenic components.

According to one aspect of the embodiment, the virus vaccine antigenic component may be based on 1) vaccine based on viral vectors; 2) a Nucleic acid vaccine (DNA or mRNA based); 3) subunit vaccines; 4) vaccine based on nanoparticles; 5) vaccines based on inactivated whole virus 6) live attenuated virus vaccines.

Yet preferably, the virus vaccine antigenic component may be based on based on a live attenuated virus vaccine generated through various methods of attenuation, including serial passage in cell lines, site directed mutagenesis, deletion of critical regions or through deoptimization of codons etc.

The term “live” is used in its conventional meaning, a live virus is a virus which has not been inactivated, i.e. a virus capable of replicating on permissive cells. A live attenuated vaccine virus is a virus which does not induce the disease caused by the corresponding wild-type virus in animals or humans and which is capable of inducing a specific immune response.

According to a preferred embodiment of the present disclosure, lyophilized/freeze-dried viral combination vaccine composition/formulation may comprise of at least three viruses consisting of live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml.

According to a preferred embodiment of the present disclosure, lyophilized/freeze-dried viral combination vaccine composition/formulation may comprise of all three viruses selected from a group consisting of live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml combined together to be given in a single shot.

As used herein, the term “Coronavirus” (CoV) belongs to family Coronaviridae, is a relatively large virus containing a single-stranded positive-sense RNA genome encapsulated within a membrane envelope with a diameter of 50-200 nm. There are four classes of coronavirus designated as alpha, beta, gamma, and delta.

The “alpha-coronavirus” class includes Canine coronavirus (CCoV); Feline coronavirus (FeCoV); Human coronavirus 229E (HCoV-229E); Porcine epidemic diarrhoea virus (PEDV); Transmissible gastroenteritis virus (TGEV); Human Coronavirus NL63 (NL or New Haven);

The “gamma-coronavirus” class includes Infectious bronchitis virus (IBV); Turkey coronavirus (Bluecomb disease virus); Pheasant coronavirus; Guinea fowl coronavirus;

The “delta-coronavirus” class includes Bulbul coronavirus (BuCoV) HKU11; Thrush coronavirus (ThCoV); Munia coronavirus (MuCoV); Porcine coronavirus (PorCov) HKU15

The “beta-coronavirus” class includes Bovine coronavirus (BCoV); Canine respiratory coronavirus (CRCoV)—Common in SE Asia and Micronesia; Human coronavirus OC43 (HCoV-OC43); Mouse hepatitis virus (MHV); Porcine haemagglutinating encephalomyelitis virus (HEV); Rat coronavirus (RCV); (HCoV-HKU1); Severe acute respiratory syndrome coronavirus (SARS-CoV); Middle East respiratory syndrome coronavirus (MERS-CoV) and the COVID-19 causative agent SARS-CoV-2. The betacoronavirus genome consists of four main structural proteins: spike (S), envelope (E), membrane (M), and nucleocapsid (N). The spike (S) protein functions as a major inducer of host immune responses. This S protein mediates host cell invasion by both SARS-CoV and SARS-CoV-2 via binding to a receptor protein called angiotensin-converting enzyme 2 (ACE2) located on the surface membrane of host cells. The S protein contains two subunits, S1 and S2. When the virus infects the virus, S1 is combined with host cell receptor ACE2, and after the protease digestion of the host cell, S1 is separated from S2, and the fusion of S2 and cell membrane is accelerated.

The small envelope protein (E) also called sM (small membrane), which is an unglycosylated trans-membrane protein of about 10 kDa, is the protein present in a smaller amount in the virion. It plays a driving role in the process of budding of coronaviruses that occurs at the level of the intermediate compartment in the endoplasmic reticulum and the Golgi apparatus.

M protein or matrix protein (25-30 kDa) is a more abundant membrane glycoprotein which is integrated into the viral particle by an M/E interaction.

N protein or nucleocapsid protein (45-50 kDa), which is the most conserved among the structural proteins of coronaviruses, is necessary to encapsidate the genomic RNA and then to direct its incorporation into the virion. This protein is also likely to be involved in RNA replication.

According to an embodiment of the present disclosure, the lyophilized/freeze-dried viral combination vaccine composition/formulation may comprise of live attenuated Coronavirus strain developed through codon de-optimization of the spike protein or codon-pair deoptimization of the spike gene or a combination of codon and codon-pair deoptimization. The Viral RNA from SARS-CoV-2, Isolate USA-WA1/2020 received from US CDC was reverse transcribed into 19 overlapping cDNA fragments. The spike gene was replaced with a deoptimized gene and the entire full-length genome is then transcribed into RNA and further transfected into Vero E6 cells. This process can be utilized to recover any variant strain of SARS-COV-2.

“SARS-CoV-2 variant” as used herein refers to a mutant form of SARS-CoV-2 that has developed naturally through the virus' replication cycles as it replicates in and/or transmits between hosts such as humans. Examples of SARS-CoV-2 variants include but are not limited to U.K. variant (also known as 20I/501Y.V1, VOC 202012/01, or B.1.1.7), South African variant (also known as 20H/501Y.V2 or B.1.351), and Brazil variant (also known as P.1), all as of 5 Sep. 2022.

U.K. variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/US A/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of 5 Sep. 2022.

Examples of the South Africa variant include but are not limited to GISAID ID Nos. EPI_ISL_766709 (hCoV-19/Sweden/20-13194/2020; 2020-12-24), EPI_ISL_768828 (hCoV-19/France/PAC-NRC2933/2020; 2020-12-22), EPI_ISL_770441 (hCoV-19/England/205280030/2020; 2020-12-24), and EPI_ISL_819798 (hCoV-19/England/OXON-F440A7/2020; 2020-12-18), all as of 5 Sep. 2022.

Examples of the Brazil variant include but are not limited to GISAID ID Nos. EPI_ISL_677212 (hCoV-19/USA/VA-DCLS-2187/2020; 2020-11-12), EPI_ISL_723494 (hCoV-19/USA/VA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPI_ISL_848197 (hCoV-19/Canada/LTRI-1258/2020; 2020-12-24), all as of 5 Sep. 2022.

The live attenuated SARS-CoV-2 strain of the invention could be the WA/1 strain, an alpha variant, delta variant, delta plus variant, beta variant, gamma variant, delta AY.3 variant, lambda variant, Omicron variant, Epsilon (B.1.427 and B.1.429); Eta (B.1.525); Iota (B.1.526); Kappa (B.1.617.1) 1.617.3; Mu (B.1.621, B.1.621.1); Zeta (P.2) or any other SARS-COV-2 strain that is at least 90% genetically similar to the WA/1 strain.

The live attenuated Coronavirus strain is obtained from Codagenix Inc, USA.

According to an embodiment of the present disclosure, the lyophilized/freeze-dried viral combination vaccine composition/formulation may comprise of live attenuated Measles virus strain derived from the Schwarz vaccine strain or Moraten strain (AF266287)—Del Valle JR, 2007 or from the Edmonston strain or Edmonston-Zagreb strain or Edmonston strain B, Moraten strain AIK-C, strain or MVbv.

According to an embodiment of the present disclosure, the lyophilized/freeze-dried viral combination vaccine composition/formulation may comprise of live attenuated Rubella virus strain derived from Rubella Virus strain RA-27/3 obtained from Dr. Stanley Plotkin, Wistar Institute, Philadelphia, USA. The RA-27/3 virus was isolated in WI-38, the human diploid cells from an explant of kidney tissue of rubella infected foetus. The virus was further attenuated by 25 serial passages in WI-38 cells.

According to an embodiment of the present disclosure, the lyophilized/freeze-dried viral combination vaccine composition/formulation may comprise of stabilizer selected from the group consisting of at least one carbohydrate, at least one amino acid and at least one hydrolyzed protein.

In accordance with the embodiments of the present disclosure, the lyophilized/freeze-dried viral combination vaccine composition/formulation may comprise of at least one carbohydrate, selected from a group consisting of, but not limited to, natural carbohydrates, synthetic carbohydrates, polyols, glass transition facilitating agents monosaccharides, disaccharides, trisaccharides, oligosaccharides and their corresponding sugar alcohols, polyhydroxyl compounds such as carbohydrate derivatives and chemically modified carbohydrates, hydroxyethyl starch and sugar copolymers. Both natural and synthetic carbohydrates are suitable for use. Synthetic carbohydrates include, but are not limited to, those which have the glycosidic bond replaced by a thiol or carbon bond. Both D and L forms of the carbohydrates may be used. The carbohydrate may be non-reducing or reducing. Where a reducing carbohydrate is used, the addition of inhibitors of the Maillard reaction is preferred. Reducing carbohydrates suitable for use in the composition are those known in the art and include, but are not limited to, glucose, sucrose, maltose, lactose, fructose, galactose, mannose, maltulose and lactulose. Non-reducing carbohydrates include, but are not limited to, non-reducing glycosides of polyhydroxyl compounds selected from sugar alcohols and other straight chain polyalcohols. Other useful carbohydrates include raffinose, stachyose, melezitose, dextran, cellibiose, mannobiose and sugar alcohols. The sugar alcohol glycosides are preferably monoglycosides, in particular the compounds obtained by reduction of disaccharides such as lactose, maltose, lactulose and maltulose. Glass forming agent is selected from the group consisting of sucrose, mannitol, trehalose, mannose, raffinose, lactitol, lactobionic acid, glucose, maltulose, iso-maltulose, maltose, lactose sorbitol, dextrose, fructose, glycerol, sorbitol, and fucose, or a combination thereof.

In an embodiment of the present disclosure, the carbohydrate may be sorbitol.

Typically, the sorbitol may be present at a concentration range of 1-20% (w/v), preferably in the range of 1-10% (w/v), and more preferably in the range of 3-6% (w/v).

Yet preferably the sorbitol may be present at a concentration of 5% (w/v).

In accordance with the embodiments of the present disclosure, the lyophilized/freeze-dried viral combination vaccine composition/formulation may comprise of at least one amino acid selected from the group, but not limited to, tricine, leucine, iso-leucine, L-histidine, glycine, glutamine, L-arginine, L-arginine hydrochloride, lysine, L-alanine, Tryptophan, Phenylalanine, Tyrosine, Valine, Cysteine, Glycine, Histidine, Methionine, Proline, Serine, Threonine, or a combination thereof.

In an embodiment of the present disclosure, the lyophilized/freeze-dried viral combination vaccine composition/formulation may comprise of amino acid selected from the group consisting of tricine, L-arginine hydrochloride, L-histidine and L-alanine as suitable amino acids individually or in combination.

The amino acid may include tricine at a concentration ranging in between 0.1% and 2% weight/volume (w/v), preferably in between 0.1-1%, more preferably in between 0.1-0.5%, most preferably equal to 0.3% (w/v).

The amino acid may include L-histidine at a concentration ranging in between 0.1% to 2% (w/v), preferably in between 0.1-1%, more preferably in between 0.1-0.5%, most preferably equal to 0.21% (w/v).

The amino acid may include L-alanine at a concentration ranging in between 0.01% and 1% weight/volume, preferably in between 0.05-0.5%, more preferably in between 0.08-0.2%, most preferably equal to 0.1% (w/v).

The amino acid may include L-arginine hydrochloride ranging in between 0.1% and 10% weight/volume, preferably in between 0.1-5%, more preferably in between 0.1-3%, most preferably equal to 1.6% (w/v).

In an embodiment of the present disclosure, the lyophilized/freeze-dried viral combination vaccine composition/formulation may comprise of at least one hydrolysed protein selected from a group consisting of gelatin, lactalbumin hydrolysate, monosodium glutamate, collagen hydrolysate, keratin hydrolysate, peptides, Casein hydrolysate and whey protein hydrolysate or protein such as serum albumin.

In another embodiment of the present disclosure, the lyophilized/freeze-dried viral combination vaccine composition/formulation may comprise of hydrolyzed protein selected from a group consisting of gelatin at a concentration ranging in between 0.1% and 10% weight/volume, preferably in between 0.1-5%, more preferably in between 0.1-3%, most preferably equal to 2.5% (w/v) and lactalbumin hydrolysate at a concentration ranging in between 0.1% and 2% weight/volume (w/v), preferably in between 0.1-1%, more preferably in between 0.1-0.5%, most preferably equal to 0.35% (w/v) individually or in combination.

As used herein, the term “gelatin” means a sterile non-pyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

The lyophilized/freeze-dried vaccine composition/formulation of the present disclosure may additionally comprise an adjuvant selected from the group of aluminum hydroxide, aluminum phosphate, aluminum hydroxyphosphate, and potassium aluminum sulfate or a mixture thereof.

The lyophilized/freeze-dried viral combination vaccine composition/formulation of the present disclosure may additionally comprise of an immunostimulatory component selected from the group consisting of Alum, an oil and water emulsion MF-59, a liposome, a lipopolysaccharide, a saponin, lipid A, lipid A derivatives, Monophosphoryl lipid A, 3-deacylated monophosphoryl lipid A, AS01, AS03, an oligonucleotide, an oligonucleotide comprising at least one unmethylated CpG and/or a liposome, Freund's adjuvant, Freund's complete adjuvant, Freund's incomplete adjuvant, polymers, co-polymers such as polyoxyethylene-polyoxypropylene copolymers, including block co-polymers, polymer p 1005, CRL-8300 adjuvant, muramyl dipeptide, TLR-4 agonists, imidazoquinolinone, Alhydroxiquim-II flagellin, flagellins derived from gram negative bacteria, TLR-5 agonists, fragments of flagellins capable of binding to TLR-5 receptors, Alpha-C-galactosylceramide, Chitosan, Interleukin-2, QS-21, ISCOMS, saponin combination with sterols and lipids.

In an embodiment of the present disclosure, the lyophilized/freeze-dried viral combination vaccine composition/formulation is in form of a single dose composition and is free of preservative. In another embodiment of the present disclosure, the lyophilized/freeze-dried viral combination vaccine composition/formulation is in form of a the multi-dose composition and the multi-dose composition may additionally comprise preservative selected from the group comprising of 2-phenoxyethanol, Benzethonium chloride (Phemerol), Phenol, m-cresol, Thiomersal, Formaldehyde, paraben esters (e.g. methyl-, ethyl-, propyl- or butyl-paraben), benzalkonium chloride, benzyl alcohol, chlorobutanol, p-chlor-m-cresol, or benzyl alcohol or a combination thereof. The lyophilized/freeze-dried viral combination vaccine composition may include material for a single immunization, or may include material for multiple immunizations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

The lyophilized/freeze-dried viral combination vaccine composition/formulation of the present disclosure may additionally comprise pharmaceutically acceptable transporter, excipient, binder, carrier, isotonic agent, emulsifier or humectant wherein pharmaceutically acceptable excipients selected from the group consisting of surfactants, polymers and salts. Examples of Surfactants may include non-ionic surfactants such as polysorbate 20, polysorbate 40, polysorbate 60, polysorbate 65, polysorbate 80, polysorbate 85, nonylphenoxypolyethoxethanol, octylphenoxypolyethoxethanol, oxtoxynol 40, nonoxynol-9, triethanolamine, triethanolamine polypeptide oleate, polyoxyethylene-660 hydroxystearate, polyoxyethylene-35 ricinoleate, soy lecithin and a poloxamer—0.001%-0.05%; polymers including dextran, carboxymethylcellulose, hyaluronic acid ad cyclodextrin. Examples of the polymers may include dextran, carboxymethylcellulose, hyaluronic acid, cyclodextrin, etc. Examples of the salts may include NaCl, KCl, KH₂PO₄, Na₂HPO₄·2H₂O, CaCl₂, MgCl₂, etc. In an embodiment of the present disclosure, the lyophilized/freeze-dried viral combination vaccine composition may be reconstituted with an aqueous solution selected from a group consisting of saline, buffer and WFI (water for injection).

In accordance with the embodiments of the present disclosure, the final pH of the reconstituted composition may be in the range of pH 6.5 to 7.5.

In accordance with the embodiments of the present disclosure, the buffering agent may be selected from a group consisting of HEPES, Citrate-phosphate, carbonate, phosphate, citrate, lactate, gluconate, borate, histidine buffer, succinate buffer and tartrate buffering agents, as well as more complex organic buffering agents including a phosphate buffering agent that contains sodium phosphate and/or potassium phosphate in a ratio selected to achieve the desired pH. In another example, the buffering agent contains Tris (hydroxymethyl) aminomethane, or “Tris”, formulated to achieve the desired pH. Yet in another example, the buffering agent could be the minimum essential medium with Hanks salts.

In accordance with the embodiments of the present disclosure, the method of manufacturing a lyophilized/freeze-dried viral combination vaccine composition may comprise of:
a) Diluting at least three viruses concentrated bulk/CMVP with a stabilizer comprising at least one carbohydrate, at least one amino acid, and at least one hydrolyzed protein to achieve the required dose per 0.5 ml;
b) Sterilizing at least three viruses bulk from step (a) by passing it through a 0.2μ-0.45μ filters;
c) Adding of Components obtained in step (b) comprising at least three viruses bulk in a blending vessel/container with agitation at room temperature;
d) Sterilizing the Components obtained in step (c) comprising at least three viruses bulk by passing it through a 0.2μ-0.45μ filters;
e) Filling into individual sterile glass vials comprising at least three viruses and partially stoppering the glass vials under aseptic conditions;
f) Freeze drying the mixture containing in the glass vials obtained in step (d) comprising the steps of freezing, sublimation and secondary drying.

One of the aspects of the embodiment, wherein the freeze drying step may comprise of
a) the freezing step may comprise freezing at −55° C. for 350 minutes to 500 minutes;
b) the sublimation step may comprise ramping at +0.5° C./minute to 1.0° C./minute to achieve a shelf temperature of −18° C., holding for 350 minutes to 500 minutes at 100 μbar; and
c) the secondary drying step may comprise ramping at +0.5° C./minute to 1.0° C./minute to achieve a shelf temperature of +23° C., holding for 350 minutes to 500 minutes at 25 μbar.

As used herein the terms “Freeze-drying” or “lyophilize” or “lyophilization” involves lyophilization and refers to the process by which a suspension is frozen, while still in the frozen state, the major portion of the water and solvent system is reduced by sublimation and secondary drying (desorption) at low pressure so as to limit biological and chemical reactions at the designated storage temperature. As used herein, the term “sublimation” refers to a change in the physical properties of a composition, wherein the composition changes directly from a solid state to a gaseous state without becoming a liquid.

One of the aspects of the embodiment, wherein the stabilizer may comprise of:
a) at least one carbohydrate is sorbitol present at a concentration of 1 to 10% (w/v);
b) at least one amino acid is selected from a group consisting of tricine present at a concentration of 0.1% to 2% (w/v), L-histidine present at a concentration of 0.1% to 2% (w/v), L-alanine present at a concentration of 0.01% to 1% (w/v) and L-arginine hydrochloride present at a concentration of 0.1% to 5% (w/v); and
c) at least one hydrolyzed protein is selected from a group consisting of gelatin present at a concentration of 0.1% to 5% (w/v) and lactalbumin hydrolysate present at a concentration of 0.10% to 2% (w/v).

Yet preferably the stabilizer may comprise of
a) carbohydrate is sorbitol present at a concentration of 5% (w/v);
b) amino acid consisting of tricine present at a concentration of 0.3% (w/v), L-histidine present at a concentration of 0.21% (w/v), L-alanine present at a concentration of 0.1% (w/v) and L-arginine hydrochloride present at a concentration of 1.6% (w/v); and
c) hydrolyzed protein consisting of gelatin present at a concentration of 2.5% (w/v) and lactalbumin hydrolysate present at a concentration of 0.35% (w/v).

According to an embodiment of the present disclosure, the live attenuated Coronavirus, live attenuated Measles virus and live attenuated Rubella virus may be passaged in a cell culture host which could be either mammalian or avian cells. Suitable mammalian cells include, but are not limited to, hamster, cattle, primate (including humans and monkeys) and dog cells. Various cell types include, but are not limited to, kidney cells, fibroblasts, retinal cells and lung cells. Examples of suitable hamster cells are the cell lines having the names BHK21 or HKCC. Suitable monkey cells are e.g. African green monkey cells, such as kidney cells as in the Vero cell line; Suitable human cells are e.g. human diploid MRC-5 cell line. Suitable dog cells are e.g. kidney cells, as in the CLDK and MDCK cell lines.

Further suitable cells include, but are not limited to: CHO; 293T; BHK; MRC 5; PER.C6; MA104 cell, BSR-T7 Cell, FRhl.2; WI-38; HeLa Cell, etc. Suitable cells are widely available e.g. from the American Type Cell Culture (ATCC) collection, from the Coriell Cell Repositories, or from the European Collection of Cell Cultures (ECACC). For example, the ATCC supplies various different Vero cells under catalogue numbers CCL 81, CCL 81.2, CRL 1586 and CRL-1587, and it supplies MDCK cells under catalogue number CCL 34. PER.C6 is available from the ECACC under deposit number 96022940.

Yet a preferred aspect of the embodiment, wherein the live attenuated Measles virus or live attenuated Rubella virus may be passaged in Human Diploid MRC-5 cells as cell culture host.

Yet a preferred aspect of the embodiment, wherein the live attenuated coronavirus may be passaged in Vero cells as cell culture host.

Vero cells (CCL-81) may be obtained from American Type Culture Collection (ATCC) will be used as cell substrate for COVID-19 Vaccine (Live, De-optimized).

Human Diploid MRC-5 cells may be obtained from National Institute of biological standards and Control (NIBSC), UK in 2003.

According to an embodiment of the present disclosure, live attenuated virus candidate may be grown onto cell culture host in adherent culture or in suspension culture mode.

Master Seed Virus may be adapted to grow in Cell culture host to prepare cell based Working Seed Virus (WSV). This cell based WSV is sub cultured and propagated in host cells using different cell culture vessels/systems like Tissue Culture Flasks (TCFs) of surface area 175 cm², Roller Bottles (RBs) of surface area 850 cm², Cell Factories (CFs) of surface area 6320 cm² and fixed-bed Bioreactor (e.g., the iCELLis® Bioreactors from Pall® Life Sciences, Port Washington, N.Y., such as the Nano and 500/100 bioreactors).

In case of Human Diploid MRC-5 cells are used as host cells for manufacturing live attenuated Measles virus or live attenuated Rubella virus, the MRC-5 cells may be cultured in Minimum essential medium (MEM) comprising 10% fetal bovine serum (FBS). Culturing of cells may occur at 37° C.±1° C. The pH value of the medium during multiplication of cells before infection may be in the range of pH 6.8 and pH 7.6 and more preferably between a value of pH 7.0 and pH 7.4.

Yet the MRC-5 cells could be cultured in serum-free or protein-free media.

In case of Vero cells are used as host cells for manufacturing live attenuated coronavirus, the vero cells may be cultured in Minimum essential medium (MEM) comprising Bovine serum albumin. Culturing of cells may occur at 37° C.±1° C. The pH value of the medium during multiplication of cells before infection may be in the range of pH 6.8 and pH 7.6 and more preferably between a value of pH 7.0 and pH 7.4. The vero cells of more than or equal to 700 million per cell factories may be used for infection of working seed virus. Not less than 5.54 Log10 PFU/0.5 mL of working seed virus may be used for infection roughly at a MOI between 1:100 to 1:10000 multiplicity of infection (moi).

According to an aspect of the embodiment, post infection with Working Seed Virus (WSV) the MRC-5 cells or vero cells may be washed with MEM without fetal bovine serum (FBS) or Bovine serum albumin and may subsequently with MEM containing protease in the range of 5 to 25 U/ml.

The protease could be selected from, however is not limited to trypsin, chymotrypsin, fungal protease, pepsin, papain, bromelain, and subtilisin.

Yet preferably the protease could be trypsin obtained from porcine origin or bovine origin or fungal origin or bacterial origin.

Yet preferably the protease could be a recombinant trypsin expressed in host cells of Yeast or Plant or Bacteria selected from but not limited to Aspergillus spp, Streptomyces griseus, Corn, E. coli, Pichia pastoris. Preferably said recombinant trypsin is selected from Biogenomics (E. coli as host), D.K. Bio Pharma Pvt. Ltd (E. coli as host), Richcore (Pichia pastoris as host) and Gibco (Fungi).

Yet the preferred trypsin concentration is 12.5 U/ml.

Yet the preferred trypsin concentration is 2000 to 3000 units of trypsin per roller bottle.

According to an aspect of the embodiment, post infection the cell supernatant may be harvested post incubation period of 40 to 78 hours; more preferably could be 48 hours and 72 hours.

Yet alternatively multiple harvesting may be carried out at an appropriate time interval of 48 hours and 72 hours for about 4-5 times before discarding the input material and processed separately to obtain clarified monovalent virus pools (CMVPs).

Purification of the live attenuated virus may be performed in single step or several steps selected from the group consisting of clarification, ultrafiltration, diafiltration or separation with chromatography.

Yet alternatively, the medium containing the virus may be clarified, typically through filters of decreasing pore sizes (e.g., 6μ, 5μ, 0.8μ, 0.65μ, 0.45μ, 0.2μ). Suitable commercially available filters and filtration devices are well known in the art and can be selected by those of skill. Exemplary filtration devices could be made of Polypropylene or Cellulose acetate or Polyethersulfone and the commercially available filters could be Millipak (Millipore), Kleenpak (Pall) and Sartobran™ P filtration devices.

In case Vero cells are used for the manufacture of bulk, additional downstream processing including non-specific endonuclease treatment and ultrafiltration may be required.

Non-specific endonuclease may be selected from Benzonase, Pulmozyme, or any other DNase and/or RNase commonly used within the art.

Harvest may be treated with a non-specific endonuclease most preferably Benzonase.

Yet alternatively the harvest may be treated with a benzonase at temperature ranging in between 30-34° C. for 1 to 6 hours, having concentration in the range of 0.5 units/ml to 6 units/ml in presence of divalent cation selected from the group consisting of Ca2+, Mg2+, Mn2+, and Cu2+ in an amount of between 0.1 mM to 100 mM.

Yet the preferred embodiment of the disclosure, wherein the filtered harvest may be treated with a benzonase at temperature of 34° C. for 2 hours having concentration of 5 to 6 units/ml in presence of divalent cation Mg²⁺ salt at concentration of between 0.1 mM to 100 mM.

The Benzonase treated harvest may be further subjected to tangential flow filtration (TFF) typically through filters with a molecular weight cut off (MWCO) ranging in between 100 KDa-500 KDa resulting in at least 10× concentration of viral harvest.

Alternatively, the Benzonase treated harvest may be further subjected to various chromatography-based purification methods.

According to an embodiment of the present disclosure, live attenuated virus candidate may be inactivated, and the methods used for virus inactivation could be heat inactivation, UV inactivation or chemical inactivation not limited to formaldehyde, beta-propiolactone etc

According to an embodiment of the present disclosure, the viral harvest may be stabilized with a stabilizer composition comprising at least one carbohydrate, at least one amino acid, and at least one hydrolyzed protein to form a stabilized viral harvest.

According to an aspect of the embodiment, the viral harvest may be stabilized with a stabilizer composition comprising:
a) at least one carbohydrate comprising sorbitol present at a concentration of 1 to 10% (w/v);
b) at least one amino acid selected from a group consisting of tricine present at a concentration of 0.1% to 2% (w/v), L-histidine present at a concentration of 0.1% to 2% (w/v), L-alanine present at a concentration of 0.01% to 1% (w/v) and L-arginine hydrochloride present at a concentration of 0.1% to 5% (w/v); and
c) at least one hydrolyzed protein selected from a group consisting of gelatin present at a concentration of 0.1% to 5% (w/v) and lactalbumin hydrolysate present at a concentration of 0.10% to 2% (w/v).

Yet preferably the viral harvest may be stabilized with a stabilizer composition comprising:
d) carbohydrate is sorbitol present at a concentration of 5% (w/v);
e) amino acid consisting of tricine present at a concentration of 0.3% (w/v), L-histidine present at a concentration of 0.21% (w/v), L-alanine present at a concentration of 0.1% (w/v) and L-arginine hydrochloride present at a concentration of 1.6% (w/v); and
f) hydrolyzed protein consisting of gelatin present at a concentration of 2.5% (w/v) and lactalbumin hydrolysate present at a concentration of 0.35% (w/v).

The stabilized viral harvest may be sterilized by DFF through at least one sterilization grade filter to obtain a Sterilized CMVPs/Virus Bulk.

According to an embodiment of the present disclosure, the stabilized viral harvest may be sterilized by direct flow filtration (DFF) through at least one sterilization grade filters preferably 0.8μ, more preferably 0.45μ, most preferably 0.2μ. Suitable commercially available filters and filtration devices are well known in the art and can be selected by those of skill. Exemplary filtration devices could be made of Polypropylene or Cellulose acetate or Polyethersulfone or Polyvinylidene difluoride and the commercially available filters could be Millipak (Millipore), Kleenpak (Pall) and Sartobran™ P filtration devices.

According to an embodiment of the present disclosure, the lyophilized/freeze-dried viral combination vaccine composition/formulation may comprise of at least one virus at a dose of not less than 1000 virus particles per 0.5 ml.

Yet preferably, the lyophilized/freeze-dried viral combination vaccine composition/formulation may comprise of live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml combined together in a single composition/formulation.

An alternative embodiment of the present disclosure, wherein the composition may be fully liquid.

According to an embodiment of the present disclosure, the lyophilized/freeze-dried viral combination vaccine composition/formulation may be formulated for use in a method for reducing the onset of or preventing a health condition comprising Coronavirus infection, Measles virus infection and Rubella virus infection involving administration of an immunologically effective amount of the combination vaccine composition to a human subject via parenteral (subcutaneous or intradermal or intramuscular or intraperitoneal or intravenous administration or injectable administration or pulmonary administration, suppositories, needle-less injection, transcutaneous) or sustained release from implants or administration by eye drops or Mucosal (oral, intranasal, pulmonary, rectal or vaginal) or buccal or peroral or intragastric or perlinqual, alveolar or gingival or olfactory or respiratory mucosa administration or interthecally, intralymphatically, via bladder instillation, or via scarification or any other routes of immunization.

According to the preferred aspect of the embodiment, the lyophilized/freeze-dried viral combination vaccine composition/formulation may be administered to a human subject via parenteral route most preferably via subcutaneous administration.

In one embodiment, it is an intranasal dispensing device, such as a device in the form of an aerosol (intranasal spray) or a drop delivery system. Liquid nasal formulations can be delivered via Instillation and rhinyle catheter, Compressed air nebulizers, Squeezed bottle, Metered-dose pump sprays like multi dose metered dose spray pumps or single/duo dose spray pump). Other dosage forms can be selected from Nasal powders (Insufflators, Dry powder inhaler), Nasal Gels, Nasal drops, Solutions, Suspensions, Cosolvent system, Microspheres, Nanoparticles, Microemulsions, Nasal insert.

The intranasal delivery devices can be selected from but not limited to Becton Dickinson (BD) Accuspray™ delivery device, Bi-Directional™ Optinose nasal device, MAD Intranasal Mucosal Atomization device by Teleflex, AeroLife™ and AeroVax™ (AerovectRx, Inc., Atlanta, GA), Jet injector—PharmaJet® Stratis®Needle-Free Injector, MUNJIs Multi-use-nozzle jet injectors: Aquapuncture device, Hypospray®, MadaJet®, GentleJet®, Disposable-syringe Jet Injectors: Medi-Jector®, J-Tip®, Injex®, Vitajet™, LectraJet HS, LectraJet® M3, ZetaJet™, PharmaJet®, Aktiv-Dry PuffHaler™ and Nasal spray flu shot device.

According to an embodiment of the present disclosure, the lyophilized/freeze-dried viral combination vaccine composition/formulation may be formulated for use in a method for reducing the onset of or preventing a health condition comprising Coronavirus virus infection or its subtypes as disclosed in earlier embodiment of the disclosure, Measles virus infection or its subtypes as disclosed in earlier embodiment of the disclosure or Rubella virus infection or its subtypes as disclosed in earlier embodiment of the disclosure.

According to a twenty first embodiment of the present disclosure, lyophilized/freeze-dried viral combination vaccine composition/formulation may be administered via parenteral route in a dose effective for the production of neutralizing antibody and meets the criterion for the seroprotection for each of the said immunogenic components comprising coronavirus, Measles virus and Rubella virus. The vaccines are administered in a manner compatible with the dosage formulation, and in such amount as will be prophylactically and/or therapeutically effective. The vaccine composition of the present disclosure can be administered as primary prophylactic agents in adults or children at the risk of infection, or can be used as secondary agents for treating infected patients. For example, the lyophilized live attenuated combination vaccine composition as disclosed herein can be used in adults or children at risk of coronavirus, Measles virus and Rubella virus infection, or can be used as secondary agents for treating coronavirus, Measles virus and Rubella virus infected patients.

More preferably the composition may be administered parenterally in a dosage volume of about 0.5 ml.

According to an embodiment of the present disclosure, the lyophilized/freeze-dried viral combination vaccine composition/formulation could be formulated as single dose vials or multidose vials or multidose kit or as pre-filled syringes or nasal sprays wherein the said lyophilized/freeze-dried vaccine composition/formulation may be given in a single dose schedule, or preferably a multiple dose schedule in which a primary course of vaccination is followed by 1-2 separate doses given at subsequent time intervals required to maintain and or reinforce the immune response, for example, at 1-4 months for a second dose, and if needed, a subsequent dose(s) after several months or years. The dosage regimen will also, at least in part, be determined on the need of a booster dose required to confer protective immunity.

Other embodiments disclosed herein encompass vaccine kit, wherein the kit may comprise of:
a) a first container containing a lyophilized/freeze-dried viral combination vaccine composition/formulation comprising: at least three viruses selected from a group consisting of live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml; carbohydrate is sorbitol present at a concentration of 1 to 10% (w/v); amino acid consisting of tricine present at a concentration of 0.1% to 2% (w/v), L-histidine present at a concentration of 0.1% to 2% (w/v), L-alanine present at a concentration of 0.01% to 1% (w/v) and L-arginine hydrochloride present at a concentration of 0.1% to 5% (w/v); and hydrolyzed protein consisting of gelatin present at a concentration of 0.1% to 5% (w/v) and lactalbumin hydrolysate present at a concentration of 0.1% to 2% (w/v); and
b) a second container containing an aqueous solution selected from saline or water for injection (WFI) for the reconstitution of the lyophilized (freeze-dried) viral combination vaccine composition.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

Throughout this specification the word, “lyophilized/freeze-dried vaccine composition/formulation” or “lyophilized live attenuated combination vaccine composition/formulation” covers any composition that elicits an immune response against the antigen or immunogen of interest; for instance, after administration into a subject, elicits an immune response against the targeted immunogen or antigen of interest. The word “lyophilized/freeze-dried vaccine composition/formulation” covers: use of single vaccine antigen or combination of more than one vaccine antigen mixed together to form a combination vaccine. The terms “vaccine composition” and “vaccine” covers any composition that induces a protective immune response against the antigen of interest, or which efficaciously protects against the antigen; for instance, after administration or injection into the subject, elicits a protective immune response against the targeted antigen or immunogen or provides efficacious protection against the antigen or immunogen.

The use of the expression “one or more” or “at least one” suggests the use of one or more elements or ingredients or quantities, as the use may be in the embodiment of the invention to achieve one or more of the desired objects or results. It may suggest comprising: one element or combination of more than one element mixed together. While certain embodiments of the inventions have been described, these embodiments have been presented by way of example only, and are not intended to limit the scope of the inventions. Variations or modifications to the composition of this invention, within the scope of the invention, may occur to those skilled in the art upon reviewing the disclosure herein. Such variations or modifications are well within the spirit of this disclosure.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary. Similarly, the components used in purification, e.g., filters, columns, are not intended to be in any way limiting or exclusionary, and can be substituted for other components to achieve the same purpose at the discretion of the practitioner.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustration of the disclosure and not as a limitation.

All patent applications/publications referred herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The corona virus strain/Covid antigen as disclosed in present patent application is one or more antigens disclosed in provisional patent applications U.S. 62/966,750 (filed on 28 Jan. 2020), U.S. 63/048,942 (filed on 7 Jul. 2020), U.S. 63/048,947 (filed on 7 Jul. 2020). U.S. 63/079,337 (filed on 16 Sep. 2020), U.S. 63/079,853 (filed on 17 Sep. 2020), and/or patent application number PCT/US2021/015246 (published as WO/2021/154828).

### Technical Advantages

The lyophilized/freeze-dried viral combination vaccine composition/formulation and method of the present disclosure described herein above has several technical advantages including, but not limited to, the realization of:
Combined immunization to Coronavirus, Measles virus and Rubella virus infectious agent in a single shot requiring a single clinic visit. Simple and effective method for inducing complete immune response to Coronavirus, Measles virus and Rubella virus infectious agent. Effective induction of systemic immune response to Coronavirus, Measles virus and Rubella virus infectious agent. Composition for effective induction of immune response to Coronavirus, Measles virus and Rubella virus infectious agent and meets the criterion for the seroprotection for each of the said immunogenic components comprising coronavirus, Measles virus and Rubella virus.
Improved immunological memory, Long-term memory cellular immune response
The lyophilized presentation of a vaccine improves stability of the vaccine composition for longer periods and the reconstituted vaccine preserves desired characteristics of a virus including virus viability, immunogenicity and stability.
Combination vaccine comprising of a live attenuated measles-rubella (MR) vaccine and a SARS CoV 2 vaccine (comprising Live Attenuated SARS-CoV-2 virus strain developed through codon de-optimization of the spike protein) may be safely administered subcutaneously such that the immunogenicity of each of the measles, rubella and SARS-CoV-2 is not inferior to that observed for each of the three viruses when administered as individual vaccines and is found to be equivalent or improved as compared to immunogenicity of SARS-CoV-2 vaccine given intranasally.
The formulation is preferably a lyophilized formulation comprising of SARS-CoV-2 vaccine with Measles-Rubella antigens along with sorbitol, gelatin, histidine, alanine, tricine, arginine & lactalbumin hydrolysate is stable, immunogenic wherein Sorbitol preserves the structural integrity (native like structure) of virus and prevents aggregation of viruses. Lactalbumin hydrolysate prevents adsorption of the viruses onto the walls of the vessel and promotes desorption done previously & Lactalbumin hydrolysate stabilizes proteins through a variety of mechanisms such as preferential hydration, direct binding, buffering, and antioxidation.
Said Combination vaccine formulation is devoid of stabilizer like sucrose, surfactants like polysorbates.
Said combination vaccine formulation can be given with or without adjuvant.

### EXAMPLES

The following examples are included to demonstrate preferred embodiments of the invention. It should be appreciated by those of skill in the art that the compositions and techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Coronavirus: The strain used for the development of COVID-19 Vaccine (Live, De-optimized), Lyophilized Injectable is CDX-005, is a Live Attenuated SARS-CoV-2 virus strain developed through codon de-optimization of the spike protein by Codagenix Inc, USA.

Viral RNA from SARS-CoV-2, Isolate IUSA-WA1/2020 received from US CDC was reverse transcribed into 19 overlapping cDNA fragments. The spike gene was replaced with a deoptimized gene which is then transcribed into RNA and further transfected into Vero E6 cells.

Measles Virus: Starting from EDMONSON B virus and going through 19 passages in human diploid cells including 3 plaquing, the EDMONSON ZAGREB MEASLES vaccine strain vas obtained. It is further attenuated strain than the EDMONSON B but retained its immunogenic properties. It was received from Institute of Immunology, Zagreb, Croatia on 15 Jul. 1989.

Rubella Virus: Rubella Virus strain RA-27/3 was obtained from Dr. Stanley Plotkin, Wistar Institute, Philadelphia, USA The virus was isolated in WI-38, the human diploid cells from an explant of kidney tissue of rubella infected foetus, The virus was further attenuated by 25 serial passages in WI-38 cells.

### Example 1: Stability Studies with Various Stabilizer Combinations

Stability studies were conducted on a coronavirus harvest with a combination of stabilizer chosen based on the process knowledge in order to select the best combination empirically. A code was assigned to each stabilizer combination. Each combination was tested for vaccine potency (using virus content as a marker) and exposed to 37±1° C. and sampling was done on day 1 and day 2, 25±1° C. and sampling done on day 1, day 3 and/or day 5, and kept at 2-8° C. and sampling done on day 15 and day 30. Virus content was estimated on the samples using plaque assay and the rate of degradation in virus content (live virus particles) were computed.

TABLE 1
Data of stability studies is shown below
with various stabilizer combinations

| | | Rate of degradation/day | | |
|---|---|---|---|---|
| Code | Stabilizer composition | 37° C. | 25 ± 5° C. | 2-8° C. |
| A | 5% Sucrose + 5% Glycine (SG) | CL | −0.42 | −0.03 |
| B | SG + 1% Human Serum Albumin | CL | −0.41 | −0.04 |
| C | 7.5% Sucrose, 1.8% phosphate, | CL | −0.40 | −0.04 |
| | 0.9% glutamate (SPG) | | | |
| D | SPG + 2% Human Serum | CL | −0.21 | −0.02 |
| | Albumin (HSA) | | | |
| E | 0.85% gelatin, 4% sucrose, | −0.76 | −0.09 | −0.01 |
| | 0.21% L-Histidine, 0.1% L-alanine, | | | |
| | 0.3% Tricine, 2.1% L-arginine | | | |
| | (GS-Stab II w/o LAH) | | | |
| F | 2.5% gelatin, 5% Sorbitol, 0.21% | −0.76 | −0.16 | −0.01 |
| | L-Histidine, 0.1% L-alanine, | | | |
| | 0.3% Tricine, 2.1% | | | |
| | L-arginine + LAH (GS-StabII) | | | |
| G | 2% Sucrose + 5% glycine | ND | −0.38 | −0.05 |
| H | 5% Glycine | ND | −0.42 | −0.04 |
| I | MEM (Bulk:MEM = 60:40) | ND | −0.31 | −0.03 |
| J | GS-StabII w/o LAH + 2% HSA | −0.77 | −0.13 | ND |
| K | GS-StabII w/o LAH + 10% FBS | −0.76 | −0.13 | ND |
| | (0.4% proteins) | | | |
| L | GS-StabII w/o LAH + 0.177% | −0.82 | −0.14 | ND |
| | Phosphate | | | |
| M | GS-StabII w/o LAH + 0.0879% | −0.79 | −0.15 | ND |
| | Glutamate | | | |
| N | GS-StabII w/o LAH (pH adjusted | −0.86 | −0.14 | ND |
| | to >7.0 with NaOH) | | | |
| E1 | GS-StabII w/o LAH in MEM | ND | −0.11 | ND |
| | (Hanks) | | | |
| E2 | GS-StabII w/o LAH in Phosphate | ND | −0.14 | ND |
| | buffered saline (PBS) | | | |
| O1 | MEM + HSA | ND | −0.27 | ND |
| O2 | PBS + HSA | ND | −0.10 | ND |
| A1 | MEM + SG | ND | −0.35 | ND |
| A2 | PBS + SG | ND | −0.38 | ND |

GS-StabII w/o LAH = 0.85% gelatin, 4% sucrose, 0.21% L-Histidine, 0.1% L-alanine, 0.3% Tricine, 2.1% L-arginine.
GS + Stab II (MMR) = 2.5% gelatin, 5% Sorbitol, 0.21% L-Histidine, 0.1% L-alanine, 0.3% Tricine, 2.1% L-arginine + LAH
Stab II = 2.5% gelatin, 5% Sorbitol, 0.21% L-Histidine, 0.1% L-alanine, 0.3% Tricine, 2.1% L-arginine
SPG = 7.5% Sucrose, 1.8% phosphate, 0.9% glutamate (7.462, 1.771, .879%)
CL = Complete loss in virus titres were observed indicating effective Rate of degradation (ROD) must be more than 7.3 Log PFU/mL (Minimum initial titer).
ND = Not done
LAH = Lactalbumin Hydrolysate

### Interpretation

Stabilizer containing combination of Gelatin and sucrose (stabilizer E & F) protected the vaccine bulk with lowest degradation rates at all temperatures. Proteins were essential to provide vaccine stability especially presence of gelatin in the stabilizer significantly protected vaccine bulk from thermal degradation especially at higher temperatures.

### Example 2: Details of Final Measles-Rubella (MR) and SARS Coronavirus Vaccine Composition

TABLE 2
SARS Coronavirus vaccine composition

| No. | Components | COVID-19 Vaccine |
|---|---|---|
| 1. | Active component | CDX-005 Not less than 3.0 log₁₀ |
| | | PFU/dose of 0.5 ml |
| 2. | Stabilizer | 2.5% Gelatin |
| | | 5% Sorbitol |
| | | 0.21% L-histidine |
| | | 0.1% L-alanine |
| | | 0.3% Tricine |
| | | 1.6% L-arginine hydrochloride |
| | | 0.35% Lactalbumin hydrolysate |

TABLE 3
Measles vaccine composition

| No. | Components | Measles Vaccine |
|---|---|---|
| 1. | Active component | Measles virus Not less than 3.0 log₁₀ |
| | | CCID₅₀/dose 0.5 ml |
| 2. | Stabilizer | 2.5% Gelatin |
| | | 5% Sorbitol |
| | | 0.21% L-histidine |
| | | 0.1% L-alanine |
| | | 0.3% Tricine |
| | | 1.6% L-arginine hydrochloride |
| | | 0.35% Lactalbumin hydrolysate |

TABLE 4
Rubella vaccine composition

| No. | Components | Rubella Vaccine |
|---|---|---|
| 1. | Active component | Rubella virus Not less than 3.0 |
| | | log₁₀ CCID₅₀/dose 0.5 ml |
| 2. | Stabilizer | 2.5% Gelatin |
| | | 5% Sorbitol |
| | | 0.21% L-histidine |
| | | 0.1% L-alanine |
| | | 0.3% Tricine |
| | | 1.6% L-arginine hydrochloride |
| | | 0.35% Lactalbumin hydrolysate |

TABLE 5
Measles-Rubella (MR) and SARS Coronavirus vaccine composition

| No. | Components | |
|---|---|---|
| 1. | Active component 1 | Measles virus Not less than 3.0 log₁₀ |
| | | CCID₅₀/dose 0.5 ml |
| | Active component 2 | Rubella virus Not less than 3.0 log₁₀ |
| | | CCID₅₀/dose 0.5 ml |
| | Active component 3 | CDX-005 Not less than 3.0 log₁₀ |
| | | PFU/dose of 0.5 ml |
| 2. | Stabilizer | 2.5% Gelatin |
| | | 5% Sorbitol |
| | | 0.21% L-histidine |
| | | 0.1% L-alanine |
| | | 0.3% Tricine |
| | | 1.6% L-arginine hydrochloride |
| | | 0.35% Lactalbumin hydrolysate |

### Example 2: Real Time Stability Data of MR-SARS Vaccine at 2-8° C. for 6 Months (M), 25° C. for 1 Month and 37° C. for 7 Days (D)

TABLE 2
Stability at 2-8° C.

| | Virus | Batch | 0 | 1 | 2 | 3 | 6 | | | Average | |
|---|---|---|---|---|---|---|---|---|---|---|---|
| | component | No. | Month | Month | Months | Months | Months | ROD/M | RSQ | degradation/M | 95% CI |
| COVID-19 | CDX-005 | 407102 | 5.46 | 5.35 | 4.85 | 5.04 | 4.75 | −0.020 | 0.72 | 0.019 | 0.016 to |
| | CDX-005 | 407103 | 5.50 | 5.35 | 4.89 | 5.07 | 4.78 | −0.018 | 0.74 | | 0.021 |
| | CDX-005 | 407104 | 5.64 | 5.45 | 5.06 | 5.22 | 4.95 | −0.018 | 0.74 | | |
| MR-COVID | CDX-005 | 500102 | 4.87 | 4.69 | 4.25 | 4.21 | 4.13 | −0.121 | 0.72 | 0.067 | 0.00 to |
| | CDX-005 | 500103 | 5.58 | 5.33 | 5.27 | 5.21 | 5.20 | −0.050 | 0.61 | | 0.162 |
| | CDX-005 | 500104 | 5.47 | 5.18 | 5.18 | 5.16 | 5.19 | −0.031 | 0.31 | | |
| | Measles | 500102 | 3.67 | 3.62 | 3.49 | 3.45 | 3.53 | −0.023 | 0.35 | −0.019 | 0.003 to |
| | Measles | 500103 | 3.63 | 3.58 | 3.62 | 3.49 | 3.49 | −0.024 | 0.67 | | 0.035 |
| | Measles | 500104 | 3.63 | 3.66 | 3.58 | 3.62 | 3.58 | −0.010 | 0.43 | | |
| | Rubella | 500102 | 3.72 | 3.70 | 3.78 | 3.62 | 3.62 | −0.020 | 0.43 | −0.015 | 0.018 to |
| | Rubella | 500103 | 3.78 | 3.83 | 3.87 | 3.66 | 3.66 | −0.029 | 0.47 | | 0.048 |
| | Rubella | 500104 | 3.83 | 3.87 | 3.83 | 3.78 | 3.87 | 0.003 | 0.03 | | |

ROD/M = Rate of degradation per month
RSQ = R square (goodness of fit)

TABLE 3
Stability at 25 ± 2° C./65 ± 5% RH

| | Virus | Batch | 0 | 1 | 2 | 3 | 6 | | | Average | |
|---|---|---|---|---|---|---|---|---|---|---|---|
| | component | No. | Months | Month | Months | Months | Months | ROD/M | RSQ | degradation/M | 95% CI |
| COVID-19 | CDX-005 | 407102 | 5.46 | 4.28 | 3.95 | 3.54 | 3.04 | −0.020 | 0.82 | 0.019 | 0.016 to |
| | CDX-005 | 407103 | 5.50 | 4.43 | 4.22 | 3.81 | 3.42 | −0.018 | 0.80 | | 0.021 |
| | CDX-005 | 407104 | 5.64 | 4.69 | 4.38 | 3.94 | 3.49 | −0.018 | 0.85 | | |
| MR-COVID | CDX-005 | 500102 | 4.87 | 3.35 | 3.14 | 2.77 | 2.12 | −0.022 | 0.72 | 0.018 | 0.011 to |
| | CDX-005 | 500103 | 5.58 | 4.59 | 4.30 | 4.16 | 3.34 | −0.015 | 0.61 | | 0.025 |
| | CDX-005 | 500104 | 5.47 | 4.56 | 4.18 | 3.93 | 3.20 | −0.017 | 0.31 | | |
| | Measles | 500102 | 3.67 | 3.03 | 2.53 | 2.49 | 2.37 | −0.013 | 0.66 | 0.012 | 0.011 to |
| | Measles | 500103 | 3.63 | 3.20 | 2.78 | 2.62 | 2.58 | −0.012 | 0.70 | | 0.013 |
| | Measles | 500104 | 3.63 | 3.37 | 2.70 | 2.70 | 2.66 | −0.012 | 0.63 | | |
| | Rubella | 500102 | 3.72 | 3.70 | 3.53 | 3.49 | 3.37 | −0.003 | 0.91 | 0.002 | 0.001 to |
| | Rubella | 500103 | 3.78 | 3.62 | 3.62 | 3.66 | 3.49 | −0.001 | 0.75 | | 0.004 |
| | Rubella | 500104 | 3.83 | 3.78 | 3.70 | 3.74 | 3.66 | −0.001 | 0.79 | | |

ROD/M = Rate of degradation per month
RSQ = R square (goodness of fit)

TABLE 4
Stability at 37 ± 1° C.

| | Virus | Batch | | | | | | | | Average | |
|---|---|---|---|---|---|---|---|---|---|---|---|
| | component | No. | 0 day | 2 days | 3 days | 5 days | 7 days | ROD/D | RSQ | degradation/D | 95% CI |
| COVID-19 | CDX-005 | 407102 | 5.25 | 4.89 | 4.77 | 4.44 | 4.22 | −0.148 | 0.99 | 0.141 | 0.127 to |
| | CDX-005 | 407103 | 5.29 | 5.00 | 4.90 | 4.52 | 4.38 | −0.135 | 0.98 | | 0.154 |
| | CDX-005 | 407104 | 5.40 | 4.94 | 4.86 | 4.56 | 4.41 | −0.139 | 0.96 | | |
| MR-COVID | CDX-005 | 500102 | 4.74 | 3.86 | 3.76 | 3.46 | 3.28 | −0.195 | 0.87 | 0.158 | 0.088 to |
| | CDX-005 | 500103 | 5.32 | 4.99 | 4.75 | 4.66 | 4.41 | −0.125 | 0.95 | | 0.228 |
| | CDX-005 | 500104 | 5.37 | 4.88 | 4.74 | 4.45 | 4.27 | −0.154 | 0.96 | | |
| | Measles | 500102 | 3.62 | 3.28 | 3.20 | 2.99 | 2.99 | −0.090 | 0.88 | 0.089 | 0.076 to |
| | Measles | 500103 | 3.58 | 3.24 | 2.99 | 2.99 | 2.87 | −0.095 | 0.83 | | 0.102 |
| | Measles | 500104 | 3.70 | 3.45 | 3.32 | 3.20 | 3.12 | −0.082 | 0.94 | | |
| | Rubella | 500102 | 3.78 | 3.70 | 3.66 | 3.62 | 3.58 | −0.028 | 0.96 | 0.026 | 0.018 to |
| | Rubella | 500103 | 3.66 | 3.53 | 3.45 | 3.45 | 3.45 | −0.028 | 0.69 | | 0.034 |
| | Rubella | 500104 | 3.78 | 3.70 | 3.66 | 3.66 | 3.62 | −0.021 | 0.86 | | |

ROD/d = Rate of degradation per month
RSQ = R square (goodness of fit)
Interpretation: Average degradation and 95% CI (upper and lower bounds) are compared above in section 4. Rubella virus component is most stable while CoV and Measles vaccines components have comparable stability profile.

TABLE 5
Stability of “MR vaccine” Vs “SARS Vaccine”
Vs “MR + SARS vaccine”

| | 2-8° C. | | 25 ± 2° C. | | 37 ± 1° C. | |
|---|---|---|---|---|---|---|
| Batch | | MR- | | MR- | | MR- |
| No. | CoV | CoV | CoV | CoV | CoV | CoV |
| Batch 1 | −0.02 | −0.121 | −0.02 | −0.022 | −0.148 | −0.195 |
| Batch 2 | −0.018 | −0.05 | −0.018 | −0.015 | −0.135 | −0.125 |
| Batch 3 | −0.018 | −0.031 | −0.018 | −0.017 | −0.139 | −0.154 |

| | | | |
|---|---|---|---|
| p-value | 0.15 | 0.78 | 0.45 |

Interpretation: No significant difference was observed in the stability profile of CDX-005 component either alone or in combination Measles-Rubella (MR)-Coronavirus (CoV) vaccine (two tailed student's t test, p < 0.05) as shown below.

### Example 3: Immunogenicity Study of “MR Vaccine” Vs “SARS Vaccine” Vs “MR+SARS Vaccine”

Comparative immunogenicity and a challenge study was conducted in 8-10 weeks old Syrian Hamsters (Mesocricetus auratus), a well established model for COVID-19, were immunized. 8 animals in each group were immunized with two doses of following vaccines 28 days apart via intramuscular route—Each group was given either MR vaccine, COVID-19 vaccine, MR vaccine or a Placebo.

TABLE 6
Comparative immunogenicity study

| S. No. | Details of immunization | DAY 1 | DAY 30 | DAY 60 | DAY 90 |
|---|---|---|---|---|---|
| 1. | MR-COVID (10\hat{ }5, IM) | <5 | 226 | 331 | 677 |
| 2. | MR-COVID (10\hat{ }4.5 IM) | <5 | <5 | 67 | 64 |
| 3. | COVID-19 vaccine | <5 | 80 | 80 | 218 |
| | (10\hat{ }5 IM) | | | | |
| 4. | COVID-19 vaccine | <5 | >1280 | 1939 | 3000 |
| | (10\hat{ }5 IN) | | | | |
| 5. | Placebo | <5 | <5 | <5 | <5 |

Interpretation: All animals were observed up to day 90 showed no loss in body weight. All animals remained healthy within the observation period. Serum samples collected on day 30, day 60 and day 90 were assessed for neutralizing antibodies by plaque reduction neutralization assay (PRNT50). The results suggest a good and a comparable immune response against COVID-19 vaccine component.

### Example 4: Comparative Virus Challenge Study of “MR Vaccine” Vs “SARS Vaccine” Vs “MR+SARS Vaccine”

Animals were challenged with wild type Coronavirus wild type Wuhan strain (10\hat{ }5.0 TCID50/animal) or Delta strain (10\hat{ }4.3 TCID50/animal) on day 90 and sacrificed on day 94. Following 90 days post challenge all animals were observed for parameters such as body weight, temperature, food intake, virus dissemination, lung gross pathology and histopathology and viral load in lungs.

TABLE 7
Comparative Virus challenge study

| | | Average viral load (TCID50/lung) | |
|---|---|---|---|
| S. No. | Details of immunization | Wuhan | Delta |
| 1. | MR-COVID (10\hat{ }5, IM) | 0.00 | 0.00 |
| 2. | MR-COVID (10\hat{ }4.5 IM) | 5.45 | 3.78 |
| 3. | COVID-19 vaccine | 0.00 | NA |
| | (10\hat{ }5 IM) | | |
| 4. | COVID-19 vaccine | 0.00 | 0.00 |
| | (10\hat{ }5 IN) | | |
| 5. | Placebo | 6.29 | 5.13 |

NA = Not included in the experimental design

Interpretation: Both MR-COVID and COVID-19 vaccines provided complete protection from COVID-19 Coronavirus challenge strains with no signs of infection by challenge viruses. Placebo group challenged with wild type strains showed generalized inflammation, oedema and local congestion in lungs. Hamsters are sensitive to wild type Coavirus infection and shows severe weight loss within 2-7 days post infection accompanied by inflammation, lesions and hemorrhage of lungs. Multifocal areas of haemorrhage were observed. However, vaccinated animal groups (MR-COVID (IM, 105 or COVID-19 vaccine (IM or IN) had normal lungs and were protected against challenged viruses. None of the vaccinated animals showed reduction in body weight unlike that of unvaccinated placebo group. Lung viral load was estimated in each group on day 94 (4 days post Challenge). Vaccinated animals were completely protected against virus spread in lungs as shown above.

### Example 5: Safety and Tolerability Data/Observations of Combination Vaccine Comprising Measles-Rubella (MR) and SARS Coronavirus Vaccine

TABLE 8
Comparative Safety and tolerability data/observations
of combination vaccine comprising Measles-Rubella
(MR) and SARS Coronavirus vaccine

| | | | Dose | |
|---|---|---|---|---|
| | | | COVID-19 | |
| Schedule | Model | Route | vaccine | MR-COVID vaccine |
| Single | Mice | Intra- | COVID-19: | COVID-19: |
| Dose | | muscular | 6.0 ± 0.5 | 6.0 ± 0.5 log10 |
| Single | Mice | Sub- | log10 | PFU/dose |
| Dose | | cutaneous | PFU/dose | Measles: |
| Single | Rats | Intra- | | 4.0 ± 0.5 log10 |
| Dose | | muscular | | CCID50/dose |
| | | | | Rubella: |
| | | | | 4.0 ± 0.5 log10 |
| | | | | CCID50/dose |
| Repeat | Hamsters | Intra- | COVID-19: | COVID-19: |
| Dose | | muscular | 6.0 ± 0.5 | 6.0 ± 0.5 log10 |
| Repeat | Rabbits | Intra- | log10 | PFU/dose |
| Dose | | muscular | PFU/dose | Measles: |
| | | | | 4.0 ± 0.5 log10 |
| | | | | CCID50/dose |
| | | | | Rubella: |
| | | | | 4.0 ± 0.5 log10 |
| | | | | CCID50/dose |

Interpretation: MR-COVID and COVID19 vaccines at a dose level of 5.0 Log PFU, a 10× human dose equivalent dose was found safe. Hamsters are very sensitive to COVID and show reduction in body weights. 8 animals in each group as shown above showed no reduction in body weights. Both vaccine formulations were safe and did not show undesirable reaction. An additional safety and tolerability studies (Pre-clinical Toxicity) with a 10× higher concentration relative to previous study is currently under way. No safety concerns have been reported till date.

### Example 6: Method of Manufacturing the Lyophilized/Freeze-Dried Vaccine Composition/Formulation

Method of manufacturing a lyophilized/freeze-dried vaccine composition/formulation comprises of:
a) Diluting at least one virus concentrated bulk with a stabilizer diluent comprising sorbitol present at a concentration of 5% (w/v); tricine present at a concentration of 0.3% (w/v), L-histidine present at a concentration of 0.21% (w/v), L-alanine present at a concentration of 0.1% (w/v) and L-arginine hydrochloride present at a concentration of 1.6% (w/v); gelatin present at a concentration of 2.5% (w/v) and lactalbumin hydrolysate present at a concentration of 0.35% (w/v) to achieve the required dose of: not less than 3 log₁₀ CCID₅₀ per 0.5 ml for live attenuated measles virus, not less than 3 log₁₀ CCID₅₀ per 0.5 ml for live attenuated rubella virus and not less than 3 log₁₀ PFU per 0.5 ml for live attenuated Coronavirus;
b) Sterilizing virus bulk from step (a) by passing it through a 0.2μ-0.45μ filter;
c) Post sterilizing adding of Components obtained in step (b) in a blending vessel/container with agitation at room temperature;
d) Sterilizing the Components obtained in step (c) by passing it through a 0.2μ-0.45μ filters;
e) Filling into individual sterile glass vials and partially stoppering the glass vials under aseptic conditions;
f) Freeze drying the mixture containing in the glass vials obtained in step (e) comprising the steps of:
   a) the freezing step comprising freezing at −55° C. for 420 min;
   b) the sublimation step comprising ramping at +0.5° C./minute to 1.0° C./minute to achieve a shelf temperature of −18° C., holding for 1600 minutes at 100 μbar; and
   c) the secondary drying step comprising ramping at +0.5° C./minute to 1.0° C./minute to achieve a shelf temperature of +23° C., holding for 420 minutes at 25 μbar.

### Example 7: Method of Manufacturing the Live Attenuated Coronavirus

The method of manufacturing live attenuated Coronavirus consist of:
a) Infecting Vero Cell culture comprising cell density 600 to 800 million per cell factories with Coronavirus at a MOI between 1:100 to 1:10000
b) Multiple harvesting of Supernatant comprising coronavirus at periodic intervals of 48 hrs and 72 hrs post incubation at 34±1° C. in MEM with Hanks salt solution;
c) Filtering the viral harvest by direct flow filtration (DFF) through at least one clarification filter having a pore size of between about 6 micrometers to about 0.45 micrometers;
d) Treating the clarified virus pool (CVP) with a non-specific endonuclease at temperature ranging in between 30-34° C. for 1 to 3 hours, wherein the non-specific endonuclease is benzonase having concentration in the range of 0.5 units/ml to 6 units/ml in presence of divalent cation selected from the group consisting of Ca2+, Mg2+, Mn2+, and Cu2+ in amount between 0.1 mM and 100 mM;
e) Concentrating the endonuclease treated CVP by tangential flow filtration (TFF) using a membrane with a molecular weight cut off (MWCO) of 100 KDa-500 KDa resulting in at least 10× concentration of viral harvest;
f) Stabilizing the TFF concentrate with a stabilizer composition comprising at least one carbohydrate is sorbitol present at a concentration of 1 to 10% (w/v); at least one amino acid selected from a group consisting of tricine present at a concentration of 0.1% to 2% (w/v), L-histidine present at a concentration of 0.1% to 2% (w/v), L-alanine present at a concentration of 0.01% to 1% (w/v) and L-arginine hydrochloride present at a concentration of 0.1% to 5% (w/v); and at least one hydrolyzed protein selected from a group consisting of gelatin present at a concentration of 0.1% to 5% (w/v) and lactalbumin hydrolysate present at a concentration of 0.1% to 2% (w/v) to form a stabilized viral harvest;
g) Sterilizing the stabilized TFF concentrate by DFF through at least one sterilization grade filter having a pore size of between about 0.8 micrometers to about 0.2 micrometers to form a sterilized Clarified Monovalent Virus Pool (CMVP).

The overall recovery of purified viruses is more than or equal to 40%.

### Example 8: Method of Manufacturing the Live Attenuated Measles Virus

The method of manufacturing live attenuated Measles virus consists of:

### Example 9: Method of Manufacturing the Live Attenuated Rubella Virus

The method of manufacturing live attenuated Rubella virus consists of:

1. A lyophilized/freeze-dried viral combination composition, comprising:
a) at least three viruses; and
b) stabilizer comprising at least one carbohydrate, at least one amino acid and at least one hydrolyzed protein,
wherein the virus is selected from any one or more of a live attenuated virus (LAV), an inactivated virus, a chimeric virus, or a recombinant virus.

2. The lyophilized/freeze-dried viral combination composition of claim 1, wherein the combination composition is a vaccine composition that preserves the desired characteristics of each virus, including stability and immunogenicity.

3. The vaccine composition according to claim 2, comprising of at least three viruses selected from any one or more of poxvirus, measles virus, mumps virus, rubella virus, sendai virus, sindbis virus and semliki forest virus (SFV), ross river virus, encephalitis virus, yellow fever virus, dengue virus, Japanese encephalitis (JE) virus, Kunjin virus, West Nile (WN) virus, tick-borne encephalitis (TBE) virus, St. Louis encephalitis virus, Murray Valley encephalitis virus, Zika virus, vesicular stomatitis virus (VSV), retrovirus, adenovirus, human adenovirus, bovine adenovirus, a canine adenovirus, a non-human primate adenovirus, a chicken adenovirus, porcine adenovirus, swine adenovirus, adeno-associated viruses, human immunodeficiency viruses (HIV), simian immunodeficiency virus (SIV), feline immunodeficiency virus (FIV)), herpes simplex virus, cytomegalovirus, Rhinovirus, Poliovirus, baculovirus vectors (autographacalifornica multiple nucleopolyhedrovirus (AcMNPV), hepatitis B virus (HBV), rubulavirus (new castle disease virus), parainfluenza virus, influenza virus, respiratory syncytial virus (RSV), human metapneumovirus (hMPV), Coronavirus (CoV), Ebola, Marburg, Nipah, Chikungunya, Rotavirus, Human papilloma virus, Herpes simplex, Hepatitis A, Hepatitis C, Hepatitis B, Hepatitis E, Variola Virus (smallpox, Monkeypox) or Varicella virus antigens.

4. The vaccine composition according to claim 3, comprising of at least three viruses selected from any one or more of live attenuated measles virus present at a dose of not less than 3 log₁₀ CCID₅₀ per 0.5 ml, live attenuated rubella virus present at a dose of not less than 3 log₁₀ CCID₅₀ per 0.5 ml, or live attenuated Coronavirus present at a dose of not less than 3 log₁₀ PFU per 0.5 ml.

5. The vaccine composition according to claim 1, comprising of at least one carbohydrate selected from any one or more of a natural carbohydrate, synthetic carbohydrate, monosaccharides, disaccharides, trisaccharides, oligosaccharides, reducing sugar, non-reducing sugar, sugar alcohols, polyol, polyhydroxyl compounds, chemically modified carbohydrates and glass transition facilitating agents which include sucrose, mannitol, trehalose, mannose, raffinose, lactitol, lactobionic acid, glucose, maltulose, iso-maltulose, maltose, lactose sorbitol, dextrose, fructose, glycerol, sorbitol, or fucose.

6. The vaccine composition according to claim 5, wherein at least one of the carbohydrate is sorbitol present at a concentration of 1 to 10% (w/v).

7. The vaccine composition according to claim 1, comprising of at least one amino acid selected from any one or more of tricine, leucine, iso-leucine, L-histidine, glycine, glutamine, L-arginine, L-arginine hydrochloride, lysine, L-alanine, Tryptophan, Phenylalanine, Tyrosine, Valine, Cysteine, Glycine, Histidine, Methionine, Proline, Serine, or Threonine.

8. The vaccine composition according to claim 6, comprising of at least one amino acid selected from any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v).

9. The vaccine composition according to claim 1, comprising of at least one hydrolyzed protein obtained by chemical, enzymatic or thermal hydrolysis of protein from either plant or animal sources.

10. The vaccine composition according to claim 8, comprising of at least one hydrolyzed protein selected from any one or more of gelatin, lactalbumin hydrolysate, monosodium glutamate, collagen hydrolysate, keratin hydrolysate, peptides, Casein hydrolysate or whey protein hydrolysate.

11. The vaccine composition according to claim 10, comprising of at least one hydrolyzed protein selected from any one or more of gelatin present at a concentration of 0.1% to 5% (w/v) or lactalbumin hydrolysate present at a concentration of 0.1% to 2% (w/v).

12. The vaccine composition according to claim 1, comprising of an adjuvant selected from any one or more of aluminum hydroxide, aluminum phosphate, aluminum hydroxyphosphate, or potassium aluminum sulfate.

13. The vaccine composition according to claim 1, comprising of an immunostimulatory component selected from any one or more of an oil and water emulsion, MF-59, a liposome, a lipopolysaccharide, a saponin, lipid A, lipid A derivatives, Monophosphoryl lipid A, 3-deacylated monophosphoryl lipid A, AS01, AS03, an oligonucleotide, an oligonucleotide comprising at least one unmethylated CpG and/or a liposome, Freund's adjuvant, Freund's complete adjuvant, Freund's incomplete adjuvant, CRL-8300 adjuvant, muramyl dipeptide, TLR-4 agonists, flagellin, flagellins derived from gram negative bacteria, TLR-5 agonists, fragments of flagellins capable of binding to TLR-5 receptors, QS-21, ISCOMS, or Chitosan, saponin combination with sterols and lipids.

14. The vaccine composition according to claim 1, comprising of a pharmaceutically acceptable additive selected from any one or more of a transporter, excipient, binder, carrier, isotonic agent, emulsifier or humectant.

15. The vaccine composition according to claim 14, wherein the excipient is selected from any one or more of a salt including NaCl, KCl, KH₂PO₄, Na₂HPO₄·2H₂O, CaCl₂, and MgCl₂; non-ionic surfactant including polysorbate 20, polysorbate 40, polysorbate 60, polysorbate 65, polysorbate 80, polysorbate 85, nonylphenoxypolyethoxethanol, octylphenoxypolyethoxethanol, oxtoxynol 40, nonoxynol-9, triethanolamine, triethanolamine polypeptide oleate, polyoxyethylene-660 hydroxystearate, polyoxyethylene-35 ricinoleate, soy lecithin and a poloxamer—0.001%-0.05%; or polymers including dextran, carboxymethylcellulose, hyaluronic acid or cyclodextrin.

16. The vaccine composition according to claim 1, wherein the lyophilized/freeze-dried viral combination vaccine composition is reconstituted with an aqueous solution selected from any one or more of a saline, buffer or water for injection.

17. The vaccine composition according to claim 16, wherein the buffer is selected from any one or more of a sodium chloride, acetate, carbonate, citrate, lactate, gluconate, tartrate, phosphate buffer saline, borate, histidine buffer, succinate buffer, HEPES, TRIS or Citrate-phosphate.

18. The vaccine composition according to claim 16, wherein the final pH of the reconstituted composition is in the range of pH 6.5 to 7.5.

19. The lyophilized/freeze-dried viral combination vaccine composition as claimed in claim 1, comprising:
a) viruses as live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml; and
b) a stabilizer comprising
a carbohydrate consisting of any one or more of sorbitol present at a concentration of 1 to 10% (w/v);
an amino acid consisting of any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and
a hydrolyzed protein consisting of any one or more of gelatin present at a concentration of 0.1% to 5% (w/v) or lactalbumin hydrolysate present at a concentration of 0.1% to 2% (w/v).

20. The lyophilized/freeze-dried viral combination vaccine composition as claimed in claim 19, comprising:
a) viruses as live attenuated measles virus present at a dose of not less than 3 log₁₀ CCID₅₀ per 0.5 ml, live attenuated rubella virus present at a dose of not less than 3 log₁₀ CCID₅₀ per 0.5 ml and live attenuated Coronavirus present at a dose of not less than 3 log₁₀ PFU per 0.5 ml; and
b) a stabilizer comprising
a carbohydrate consisting of any one or more of sorbitol present at a concentration of 5% (w/v);
an amino acid consisting of any one or more of tricine present at a concentration of 0.3% (w/v), L-histidine present at a concentration of 0.21% (w/v), L-alanine present at a concentration of 0.1% (w/v) or L-arginine hydrochloride present at a concentration of 1.6% (w/v); and
a hydrolyzed protein consisting of gelatin present at a concentration of 2.5% (w/v) and lactalbumin hydrolysate present at a concentration of 0.35% (w/v).

21. A method of manufacturing the lyophilized/freeze-dried viral combination vaccine composition of claim 1, the method comprising:
a) diluting at least three virus concentrated bulk with a stabilizer comprising at least one carbohydrate, at least one amino acid, and at least one hydrolyzed protein to achieve the required dose per 0.5 ml, wherein at least three virus concentrated bulk is selected from any one or more of a live attenuated measles virus, live attenuated rubella virus or live attenuated coronavirus;
b) sterilizing the at least three virus bulk from step (a) by passing it through a 0.2μ-0.45μ filters;
c) adding the sterilized at least three virus bulk obtained in step (b) in a blending vessel/container and agitating at room temperature;
d) sterilizing the agitated at least three virus bulk obtained in step (c) by passing it through a 0.2μ-0.45μ filters;
e) Filling the sterilized, agitated at least three virus bulk obtained in step d) into individual sterile glass vials and partially stoppering the glass vials under aseptic conditions; and
f) Freeze drying a mixture in the glass vials obtained in step (e) comprising the steps of freezing, sublimation and secondary drying.

22. The method according to claim 21, wherein the freeze drying step comprises in:
a) the freezing step comprising freezing at −55° C. for 350 minutes to 500 minutes;
b) the sublimation step comprising ramping at +0.5° C./minute to 1.0° C./minute to achieve a shelf temperature of −18° C., holding for 350 minutes to 500 minutes at 100 bar; and
c) the secondary drying step comprising ramping at +0.5° C./minute to 1.0° C./minute to achieve a shelf temperature of +23° C., holding for 350 minutes to 500 minutes at 25 bar.

23. The method according to claim 21, the stabilizer comprising:
a) a carbohydrate consisting of sorbitol present at a concentration of 1 to 10% (w/v);
b) an amino acid consisting of any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and
c) a hydrolyzed protein consisting of gelatin present at a concentration of 0.1% to 5% (w/v) and lactalbumin hydrolysate present at a concentration of 0.1% to 2% (w/v).

24. A live attenuated Coronavirus according to claim 19, obtained by a process comprising the steps of:
a) Infecting Vero Cell culture comprising cell density 600 to 800 million per cell factories with Coronavirus at a MOI between 1:100 to 1:10000;
b) Multiple harvesting of Supernatant comprising coronavirus at periodic intervals of 48 hrs and 72 hrs post incubation at 34±1° C. in MEM with Hanks salt solution;
c) Filtering the viral harvest by direct flow filtration (DFF) through at least one clarification filter having a pore size of between about 6 micrometers to about 0.45 micrometers;
d) Treating the clarified virus pool (CVP) with a non-specific endonuclease at temperature ranging in between 30-34° C. for 1 to 3 hours, wherein the non-specific endonuclease is benzonase having concentration in the range of 0.5 units/ml to 6 units/ml in presence of divalent cation selected from the group consisting of Ca2+, Mg2+, Mn2+, and Cu2+ in amount between 0.1 mM and 100 mM;
e) Concentrating the endonuclease treated CVP by tangential flow filtration (TFF) using a membrane with a molecular weight cut off (MWCO) of 100 KDa-500 KDa resulting in at least 10× concentration of viral harvest;
f) Stabilizing the TFF concentrate with a stabilizer composition comprising a carbohydrate consisting of sorbitol present at a concentration of 1 to 10% (w/v); an amino acid consisting of any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and a hydrolyzed protein consisting of any one or more of a gelatin present at a concentration of 0.1% to 5% (w/v) or lactalbumin hydrolysate present at a concentration of 0.1% to 2% (w/v) to form a stabilized viral harvest; and
g) Sterilizing the stabilized TFF concentrate by DFF through at least one sterilization grade filter having a pore size of between about 0.8 micrometers to about 0.2 micrometers to form a sterilized Clarified Monovalent Virus Pool (CMVP),
wherein the overall recovery of purified viruses is more than or equal to 40%.

25. The process according to claim 24, wherein the live attenuated Coronavirus are propagated in Vero Cells CCL-81 obtained from American Type Culture Collection (ATCC).

26. A live attenuated measles virus according to claim 19, obtained by a process comprising the steps of:
a) Infecting MRC-5 Cell culture comprising cell density 600 to 800 million per cell factories with Measles virus at a MOI between 1:100 to 1:10000;
b) Multiple harvesting of Supernatant comprising measles virus at periodic intervals of 48 hrs and 72 hrs post incubation at 34±1° C. in MEM without FBS;
c) Filtering the viral harvest by direct flow filtration (DFF) through at least one clarification filter having a pore size of between about 6 micrometers to about 0.45 micrometers;
d) Stabilizing the viral harvest with a stabilizer composition comprising a carbohydrate consisting of sorbitol present at a concentration of 1 to 10% (w/v); an amino acid consisting of any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and a hydrolyzed protein consisting of any one or more of gelatin present at a concentration of 0.1% to 5% (w/v) or lactalbumin hydrolysate present at a concentration of 0.1% to 2% (w/v) to form a stabilized viral harvest; and
e) Sterilizing the stabilized viral harvest by DFF through at least one sterilization grade filter having a pore size of between about 0.8 micrometers to about 0.2 micrometers to form a sterilized Clarified Monovalent Virus Pool (CMVP),
wherein the overall recovery of purified viruses is more than or equal to 40%.

27. The process according to claim 26, wherein the live attenuated measles virus are propagated in MRC-5 Cell PDL-7 obtained from National Institute of biological standards and Control (NIBSC), UK.

28. A live attenuated rubella virus according to claim 19, obtained by a process comprising the steps of:
a) Surface Infection of MRC-5 Cell culture comprising cell density 600 to 800 million per cell factories with rubella virus at a MOI between 1:100 to 1:10000;
b) Multiple harvesting of Supernatant comprising rubella virus at periodic intervals of 48 hrs and 72 hrs post incubation at 34±1° C. in MEM without FBS;
c) Filtering the viral harvest by direct flow filtration (DFF) through at least one clarification filter having a pore size of between about 6 micrometers to about 0.45 micrometers;
d) Stabilizing the viral harvest with a stabilizer composition comprising carbohydrate consisting of any one or more of sorbitol present at a concentration of 1 to 10% (w/v); an amino acid consisting of any one or more of tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and hydrolyzed protein consisting of any one or more of gelatin present at a concentration of 0.1% to 5% (w/v) or lactalbumin hydrolysate present at a concentration of 0.1% to 2% (w/v) to form a stabilized viral harvest; and
e) Sterilizing the stabilized viral harvest by DFF through at least one sterilization grade filter having a pore size of between about 0.8 micrometers to about 0.2 micrometers to form a sterilized Clarified Monovalent Virus Pool (CMVP),
wherein the overall recovery of purified viruses is more than or equal to 40%.

29. The process according to claim 26, wherein the live attenuated rubella virus are propagated in MRC-5 Cell PDL-7 obtained from National Institute of biological standards and Control (NIBSC), UK.

30. A kit comprising:
a) a first container containing a lyophilized (freeze-dried) viral combination vaccine composition comprising: a virus consisting of any one or more of live attenuated measles virus present at a dose of not less than 3 log₁₀ CCID₅₀ per 0.5 ml, live attenuated rubella virus present at a dose of not less than 3 log₁₀ CCID₅₀ per 0.5 ml or live attenuated Coronavirus present at a dose of not less than 3 log₁₀ PFU per 0.5 ml; a carbohydrate consisting of sorbitol present at a concentration of 1 to 10% (w/v); an amino acid consisting of any one or more tricine present at a concentration of 0.1% to 2% (w/v), L-histidine present at a concentration of 0.1% to 2% (w/v), L-alanine present at a concentration of 0.01% to 1% (w/v) or L-arginine hydrochloride present at a concentration of 0.1% to 5% (w/v); and a hydrolyzed protein consisting of any one or more of gelatin present at a concentration of 0.1% to 5% (w/v) or lactalbumin hydrolysate present at a concentration of 0.1% to 2% (w/v); and
b) a second container containing an aqueous solution selected from any one or more of saline or water for injection (WFI) for the reconstitution of the lyophilized (freeze-dried) vaccine composition.
